(19) United States
(12) Patent Application Publication
(10) Pub. No.: US 2005/0244813 A1
(43) Pub. Date: Nov. 3, 2005

(54) Detection of human papillomavirus e6 mrna

(76) Inventor: Frank Karlsen, Klokkarstua (NO)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/500,831

(22) PCT Filed: Jan. 7, 2003
(86) PCT No.: PCT/GB03/00030

§ 371 Date: Jul. 7, 2004

(30) Foreign Application Priority Data
Jan. 7, 2002 (GB) 0200258.2

(51) Int. Cl. C12Q 1/70; C12Q 1/68; C07H 21/02

(52) U.S. Cl. 435/5; 435/6; 536/23.72

(57) ABSTRACT

An oligonucleotide molecule for use in the detection of mRNA transcribed from the E6 gene of a human papillomavirus, the oligonucleotide comprising any one of sequence numbers 1-133.

The present invention is concerned with oligonucleotide primers and probes for use in detecting the presence of mRNA transcripts from the E6 gene of human papillomavirus in clinical samples.

In the last few years, there has been an improvement in the methods used to detect HPV, with methods based on amplification of nucleic acids using the polymerase chain reaction (PCR) becoming increasingly widespread. It is now possible to detect small amounts of HPV DNA (<100 pg), quantify the amount of viral DNA in clinical samples, identify a broad spectrum of genital HPV types, test for selected HPV types and localise the viral genome transcripts and proteins to the individual cells. Since HPV detection is often carried out in the presence of vast quantities of host nucleic acids and cells not infected with the virus, the ability of the primers to be virus specific is critical for a sensitive and specific amplification.

The present inventors have selected new primer and probe sequences, specific for the E6 region, which may be used in the detection of E6 transcripts by the NASBA technique, particularly sensitive, real-time NASBA, or by RT-PCR. The inventors' approach is based upon the development of primers specific for regions of E6 which are conserved across high-risk, cancer-associated HPV types.

Therefore, in accordance with a first aspect the invention provides target-specific primers and oligonucleotide probes for use in the detection of human papillomavirus (HPV) E6 mRNA, particularly for use in detection of HPV E6 mRNA by RT-PCR or NASBA. In particular, the invention provides primer and probe oligonucleotides-comprising the HPV-specific sequences represented as sequence numbers (SEQ NO) 1 to 133 in Table 1. For each individual sequence an indication is given in the column “primer/probe type” of the general types of primers or probes into which the HPV-specific sequence may be incorporated for the purposes of HPV detection. The HPV type and position in the HPV genome is also indicated.

TABLE 1

Summary of primer sequences

| | | | | |
|---|---|---|---|---|
| PRIMER/PROBE | | SEQ | | |
| TYPE | SEQUENCE | NO | HPV | nt |

| | | | | |
|---|---|---|---|---|
| NASBA P2/PCR | CCACAGGAGCGACCCAGAAAGTTA | 1 | 16 | 116 |
| NASBA P1/PCR | ACGGTTTGTTGTATTGCTGTTC | 2 | 16 | 368 |
| NASBA P2/PCR | CCACAGGAGCGACCCAGAAA | 3 | 16 | 116 |
| NASBA 21/PCR | GGTTTGTTGTATTGCTGTTC | 4 | 16 | 368 |
| NASBA P1/PCR | TCACGTCGCAGTAACTGT | 126 | 16 | 208 |
| NASBA P1/PCR | TTGCTTGCAGTACACACA | 127 | 16 | 191 |
| NASBA P1/PCR | TGCAGTACACACATTCTA | 128 | 16 | 186 |
| NASBA P1/PCR | GCAGTACACACATTCTAA | 129 | 16 | 185 |
| NASBA P2/PCR | ACAGTTATGCACAGAGCT | 130 | 16 | 142 |
| PROBE | | | | |
| NASBA P2/PCR | ATATTAGAATGTGTGTAC | 131 | 16 | 182 |
| PROBE | | | | |
| NASBA P2/PCR | TTAGAATGTGTGTACTGC | 132 | 16 | 185 |
| PROBE | | | | |
| NASBA P2/PCR | AATGTGTGTACTGCAAG | 133 | 16 | 188 |
| PROBE | | | | |
| PROBE | CTTTGCTTTTCGGGATTTATGC | 5 | 16 | 235 |
| PROBE | TATGACTTTGCTTTTCGGGA | 6 | 16 | 230 |
| NASBA P2/PCR | CAGAGGAGGAGGATGAAATAGTA | 7 | 16 | 656 |
| NASBA P1/PCR | GCACAACCGAAGCGTAGAGTCACAC | 8 | 16 | 741 |
| PROBE | TGGACAAGCAGAACCGGACAGAGC | 9 | 16 | 687 |
| NASBA P2/PCR | CAGAGGAGGAGGATGAAATAGA | 10 | 16 | 656 |
| NASBA P1/PCR | GCACAACCGAAGCGTAGAGTCA | 11 | 16 | 741 |
| PROBE | AGCAGAACCGGACAGAGCCCATTA | 12 | 16 | 693 |
| NASBA P2/PCR | ACGATGAAATAGATGGAGTT | 13 | 18 | 702 |
| NASBA P1/PCR | CACGGACACACAAAGGACAG | 14 | 18 | 869 |
| PROBE | AGCCGAACCACAACGTCACA | 15 | 18 | 748 |
| NASBA P2/PCR | GAAAACGATGAAATAGATGGAG | 16 | 18 | 698 |
| NASBA P1/PCR | ACACCACGGACACACAAAGGACAG | 17 | 18 | 869 |
| PROBE | GAACCACAACGTCACACAATG | 18 | 18 | 752 |
| NASBA P2/PCR | TTCCGGTTGACCTTCTATGT | 19 | 18 | 651 |
| NASBA P1/PCR | GGTCGTCTGCTGAGCTTTCT | 20 | 18 | 817 |
| NASBA P2/PCR | GCAAGACATAGAAATAACCTG | 21 | 18 | 179 |
| NASBA P1/PCR | ACCCAGTGTTAGTTAGTT | 22 | 18 | 379 |
| PROBE | TGCAAGACAGTATTGGAACT | 23 | 18 | 207 |
| NASBA P2/PCR | GGAAATACCCTACGATGAAC | 24 | 31 | 164 |
| NASBA P1/PCR | GGACACAACGGTCTTTGACA | 25 | 31 | 423 |
| PROBE | ATAGGGACGACACACCACACGGAG | 26 | 31 | 268 |
| NASBA P2/PCR | GGAAATACCCTACGATGAACTA | 27 | 31 | 164 |
| NASBA P1/PCR | CTGGACACAACGGTCTTTGACA | 28 | 31 | 423 |
| PROBE | TAGGGACGACACACCACACGGA | 29 | 31 | 269 |
| NASBA P2/PCR | ACTGACCTCCACTGTTATGA | 30 | 31 | 617 |
| NASBA P1/PCR | TATCTACTTGTGTGCTCTGT | 31 | 31 | 766 |
| PROBE | GACAAGCAGAACCGGACACATC | 32 | 31 | 687 |
| NASBA P2/PCR | TGACCTCCACTGTTATGAGCAATT | 33 | 31 | 619 |
| NASBA P1/PCR | TGCGAATATCTACTTGTGTGCTCTGT | 34 | 31 | 766 |
| PROBE | GGACAAGCAGAACCGGACACATCCAA | 35 | 31 | 686 |
| NASBA P2/PCR | ACTGACCTCCACTGTTAT | 36 | 31 | 617 |
| NASBA P1/PCR | CACGATTCCAAATGAGCCCAT | 37 | 31 | 809 |
| NASBA P2/PCR | TATCCTGAACCAACTGACCTAT | 38 | 33 | 618 |
| NASBA P1/PCR | TTGACACATAAACGAACTG | 39 | 33 | 763 |
| PROBE | CAGATGGACAAGCACAACC | 40 | 33 | 694 |
| NASBA P2/PCR | TCCTGAACCAACTGACCTAT | 41 | 33 | 620 |
| NASBA P1/PCR | CCCATAAGTAGTTGCTGTAT | 42 | 33 | 807 |
| PROBE | GGACAAGCACAACCAGCCACAGC | 43 | 33 | 699 |
| NASBA P2/PCR | GACCTTTGTGTCCTCAAGAA | 44 | 33 | 431 |
| NASBA P1/PCR | AGGTCAGTTGGTTCAGGATA | 45 | 33 | 618 |
| PROBE | AGAAACTGCACTGTGACGTGT | 46 | 33 | 543 |
| NASBA P2/PCR | ATTACAGCGGAGTGAGGTAT | 47 | 35 | 217 |
| NASBA P1/PCR | GTCTTTGCTTTTCAACTGGA | 48 | 35 | 442 |
| NASBA P2/PCR | TCAGAGGAGGAGGAAGATACTA | 49 | 35 | 655 |
| NASBA P1/PCR | GATTATGCTCTCTGTGAACA | 50 | 35 | 844 |
| NASBA P2/PCR | CCCGAGGCAACTGACCTATA | 51 | 35 | 610 |
| NASBA P1/PCR | GTCAATGTGTGTGCTCTGTA | 52 | 35 | 770 |
| PROBE | ATAGAGAAGGCCAGCCATAT | 53 | 35 | 270 |
| PROBE | GACAAGCAAAACCAGACACCTCCAA | 54 | 35 | 692 |
| PROBE | GACAAGCAAAACCAGACACC | 55 | 35 | 692 |
| NASBA P2/PCR | TTGTGTGAGGTGCTGGAAGAAT | 56 | 52 | 144 |
| NASBA P1/PCR | CCCTCTCTTCTAATGTTT | 57 | 52 | 358 |
| PROBE | GTGCCTACGCTTTTTATCTA | 58 | 52 | 296 |
| NASBA P2/PCR | GTGCCTACGCTTTTTATCTA | 59 | 52 | 296 |
| NASBA P1/PCR | GGGGTCTCCAACACTCTGAACA | 60 | 52 | 507 |
| PROBE | TGCAAACAAGCGATTTCA | 61 | 52 | 461 |
| NASBA P2/PCR | TCAGGCGTTGGAGACATC | 62 | 58 | 157 |
| NASBA P1/PCR | AGCAATCGTAAGCACACT | 63 | 58 | 301 |
| NASBA P2/PCR | TCTGTGCATGAAATCGAA | 64 | 58 | 173 |
| NASBA P1/PCR | AGCACACTTTACATACTG | 65 | 58 | 291 |
| PROBE | TGAAATGCGTTGAATGCA | 66 | 58 | 192 |
| PROBE | TTGCAGCGATCTGAGGTATATG | 67 | 58 | 218 |
| NASBA P2/PCR | TACACTGCTGGACAACAT | 68 | B | 514 |
| NASBA P1/PCR | TCATCTTCTGAGCTGTCT | 69 | B | 619 |
| NASBA P2/PCR | TACACTGCTGGACAACATGCA | 70 | B | 514 |
| NASBA P1/PCR | GTCACATCCACAGCAACAGGTCA | 71 | B | 693 |
| PROBE | GTAGGGTTACATTGCTATGA | 72 | B | 590 |
| PROBE | GTAGGGTTACATTGCTATGAGC | 73 | B | 590 |
| NASBA P2/PCR | TGACCTGTTGCTGTGGATGTGA | 74 | B | 693 |
| NASBA P1/PCR | TACCTGAATCGTCCGCCAT | 75 | B | 832 |
| PROBE | ATWGTGTGTCCCATCTGC | 76 | B | 794 |
| NASBA P2/PCR | CATGCCATAAATGTATAGA | 77 | C | 295 |
| NASBA P1/PCR | CACCGCAGGCACCTTATTAA | 78 | C | 408 |
| PROBE | AGAATTAGAGAATTAAGA | 79 | C | 324 |
| NASBA P2/PCR | GCAGACGACCACTACAGCAAA | 80 | 39 | 210 |
| NASBA P1/PCR | ACACCGAGTCCGAGTAATA | 81 | 39 | 344 |
| PROBE | ATAGGGACGGGGAACCACT | 82 | 39 | 273 |
| NASBA P2/PCR | TATTACTCGGACTCGGTGT | 83 | 39 | 344 |
| NASBA P1/PCR | CTTGGGTTTCTCTTCGTGTTA | 84 | 39 | 558 |
| PROBE | GGACCACAAAACGGGAGGAC | 85 | 39 | 531 |
| NASBA P2/PCR | GAAATAGATGAACCCGACCA | 86 | 39 | 703 |
| NASBA P1/PCR | GCACACCACGGACACACAAA | 87 | 39 | 886 |
| PROBE | TAGCCAGACGGGATGAACCACAGC | 88 | 39 | 749 |
| NASBA P2/PCR | AACCATTGAACCCAGCAGAAA | 89 | 45 | 430 |
| NASBA P1/PCR | TCTTTCTTGCCGTGCCTGGTCA | 90 | 45 | 527 |
| NASBA P2/PCR | GAAACCATTGAACCCAGCAGAAAA | 91 | 45 | 428 |
| NASBA P1/PCR | TTGCTATACTTGTGTTTCCCTACG | 92 | 45 | 558 |
| PROBE | GTACCGAGGGCAGTGTAATA | 93 | 45 | 500 |
| PROBE | GGACAAACGAAGATTTCACA | 94 | 45 | 467 |
| NASBA P2/PCR | GTTGACCTGTTGTGTTACCAGCAAT | 95 | 45 | 656 |
| NASBA P1/PCR | CACCACGGACACACAAAGGACAAG | 96 | 45 | 868 |
| NASBA P2/PCR | CTGTTGACCTGTTGTGTTACGA | 97 | 45 | 654 |
| NASBA P1/PCR | CCACGGACACACAAAGGACAAG | 98 | 45 | 868 |
| NASBA P2/PCR | GTTGACCTGTTGTGTTACGA | 99 | 45 | 656 |
| NASBA P1/PCR | ACGGACACACAAAGGACAAG | 100 | 45 | 868 |
| PROBE | GAGTCAGAGGAGGAAAACGATG | 101 | 45 | 686 |
| PROBE | AGGAAAACGATGAAGCAGATGGAGT | 102 | 45 | 696 |
| PROBE | ACAACTACCAGCCCGACGAGCCGAA | 103 | 45 | 730 |
| NASBA P2/PCR | GGAGGAGGATGAAGTAGATA | 104 | 51 | 658 |
| NASBA P1/PCR | GCCCATTAACATCTGCTGTA | 105 | 51 | 807 |
| NASBA P2/PCR | AGAGGAGGAGGATGAAGTAGATA | 106 | 51 | 655 |
| NASBA P1/PCR | ACGGGCAAACCAGGCTTAGT | 107 | 51 | 829 |
| PROBE | GCAGGTGTTCAAGTGTAGTA | 108 | 51 | 747 |
| PROBE | TGGCAGTGGAAAGCAGTGGAGACA | 109 | 51 | 771 |
| NASBA P2/PCR | TTGGGGTGCTGGAGACAAACATCT | 110 | 56 | 519 |
| NASBA P1/PCR | TTCATCCTCATCCTCATCCTCTGA | 111 | 56 | 665 |
| NASBA P2/PCR | TGGGGTGCTGGAGACAAACATC | 112 | 56 | 520 |
| NASBA P1/PCR | CATCCTCATCCTCATCCTCTGA | 113 | 56 | 665 |
| NASBA P2/PCR | TTGGGGTGCTGGAGACAAACAT | 114 | 56 | 519 |
| NASBA P1/PCR | CCACAAACTTACACTCACAACA | 115 | 56 | 764 |
| PROBE | AAAGTACCAACGCTGCAAGACGT | 116 | 56 | 581 |
| PROBE | AGAACTAACACCTCAAACAGAAAT | 117 | 56 | 610 |
| PROBE | AGTACCAACGCTGCAAGACGTT | 118 | 56 | 583 |
| PROBE | TTGGACAGCTCAGAGGATGAGG | 119 | 56 | 656 |
| NASBA P2/PCR | GATTTTCCTTATGCAGTGTG | 120 | 56 | 279 |
| NASBA P1/PCR | GACATCTGTAGCACCTTATT | 121 | 56 | 410 |
| PROBE | GACTATTCAGTGTATGGAGC | 122 | 56 | 348 |
| PROBE | CAACTGAYCTMYACTGTTATGA | 123 | A | |
| PROBE | GAAMCAACTGACCTAYWCTGCTAT | 124 | A | |
| PROBE | AAGACATTATTCAGACTC | 125 | A | |

Oligonucleotides for use as NASBA P1 primers have the general structure “X₁-SEQ”, wherein “X₁” represents a nucleotide sequence comprising a promoter and “SEQ” represents the HPV-specific sequence, as given in Table 1. The inclusion of a promoter sequence is essential in NASBA P1 primers but is not necessary in PCR primers, as discussed below. In a preferred embodiment, X₁ may be a sequence comprising a bacteriophage promoter, preferably the T7 promoter. In the most preferred embodiment, X₁ represents the sequence AATTCTAATACGACTCACTATAGGGAGAAGG.

The oligonucleotide molecules of the invention are selected to be specific for mRNA transcribed from the HPV E6 gene. Active expression of the E7 and E6 genes of HPV is associated with cervical cytological abnormalities which often progress to more serious disease. A number of studies relate the expression of the E6 and E7 genes to oncogenesis. Co-operation between E6 and E7 increases significantly the frequency of immortalization. Evidence has been presented that the E6 and E7 open reading frames are involved in the transforming activity of the virus (Tanaka et al., J. Virol. 63: 1465-1469, 1989). These transformation effects of E6 and E7 may at least in part be explained by their interaction with the cellular tumour suppressor gene products p53 and pRb 105, respectively (Boyer et al., Cancer Research. 56: 4620-4624, 1996; Lechner et al. EMBO J. 11: 3045-3051, 1992).

HPV16 mRNA isolated from transfected cells and a variety of tumour cell lines and lesions containing both extrachromosomal and integrated HPV16 genomes has been analysed in multiple laboratories (see Doorbar J A et al., Virology 178:254?262, Rohlfs et al., Virology 183:331?342; Sherman et al., Int. J. Cancer 50:356?364). These studies have shown that several different alternatively spliced transcripts may be produced from the E6 and E7 region. In summary, there are four major transcripts: one with the whole E6/E7 gene area (E6), one with a loss of a coding sequence between basepairs 226 and 409 (E6*I), one with a loss of a coding sequence in a larger part of E6 between 226 and 526 (E6*II) and one with the loss of the E7 transcript (E6*III). However there are clearly consensus sequences in the area up to 226 basepairs in the E6 region. The inventors therefore selected the areas between 97 and 226 and between 526 and 880 as areas to target for diagnostic purposes.

The oligonucleotides provided by the invention may be grouped according to specificity for different specific HPV types or groups of HPV types. Sequence numbers 1-12 and 126-133 are specific for HPV type 16, sequence numbers 13-23 are specific for HPV type 18, sequence numbers 24-37 are specific for HPV type 31, sequence numbers 38-46 are specific for HPV type -33. HPV types 16, 18 , 31 and 33 are the major cancer-associated HPV types. Sequence numbers 47-55 are specific for HPV type 35, sequence numbers 56-61 are specific for HPV type 52, sequence numbers 62-67 are specific for HPV type 58, sequence numbers 80-88 are specific for HPV type 39, sequence numbers 89-103 are specific for HPV type 45, sequence numbers 104-109 are specific for HPV type 51, sequence numbers 110-122 are specific for HPV type 56. Sequence numbers 68-76 are consensus sequences for group B HPV types (in particular HPV types 6 and 11). Sequence numbers 77-79 and 125 are consensus sequences for group C HPV types (including HPV types 18, 39 and 45). Sequence numbers 123 and 124 are consensus probe sequences for group A HPV types. Sequence 123 is a consensus for HPV types 16, 31 and 35; sequence 124 is a consensus for HPV types 33, 52 and 58).

The oligonucleotide molecules of the invention are preferably single stranded DNA molecules. Non-natural synthetic polynucleotides which retain the ability to base-pair with a complementary nucleic acid molecule and are also within the scope of the invention, including synthetic oligonucleotides which incorporate modified bases and synthetic oligonucleotides wherein the links between individual nucleosides include bonds other than phosphodiester bonds. The oligonucleotide molecules of the invention may be produced according to techniques well known in the art, such as by chemical synthesis using standard apparatus and protocols for oligonucleotide synthesis.

The oligonucleotide molecules provided by the invention will typically be isolated single-stranded polynucleotides of no more than 100 bases in length, more typically less than 55 bases in length. For the avoidance of doubt it is hereby stated that the language “oligonucleotide comprising sequence number n” excludes the naturally occurring full-length HPV genomes.

The invention provides several general types of oligonucleotide primers and probes incorporating the HPV-specific sequences listed in Table 1. Typically, such oligonucleotides may comprise additional, non-HPV sequences, for example sequences which are required for an amplification reaction or which facilitate detection of the products of the amplification reaction. The HPV-specific part of the oligonucleotide may consist of one of the sequences listed in Table 1 in the absence of any other contiguous HPV sequences. However, it will be appreciated that minor variations may be made to the HPV-specific sequences, for example the addition, deletion or substitution of bases, without affecting the ability of the oligonucleotide to bind to its target sequence and function as a primer or probe to a material extent.

The first type of oligonucleotides are primer 1 oligonucleotides (also referred to herein as NASBA P1 primers), which are oligonucleotides of generally approximately 50 bases in length, containing an average of about 20 bases at the 3′ end that are complementary to a region of the target mRNA. Oligonucleotides suitable for use as NASBA P1 primers are denoted “NASBA P1/PCR” in Table 1. The 5′ ends of the P1 primer oligonucleotides (represented herein in general terms as X₁) comprise a promoter sequence that is recognized by a specific RNA polymerase. Bacteriophage promoters, for example the T7, T3 and SP6 promoters, are preferred for use in the oligonucleotides of the invention, since they provide advantages of high level transcription which is dependent only on binding of the appropriate RNA polymerase. In a preferred embodiment, the 5′ terminal sequence of the P1 primer oligonucleotides may comprise the sequence AATTCTAATACGACTCACTATAGGG or the sequence AATTCTAATACGACTCACTATAGGGAGAAGG. These sequences contains a T7 promoter, including the transcription initiation site for T7 RNA polymerase. The HPV-specific sequences denoted in Table 1 as “NASBA P1/PCR” are suitable for use in both NASBA P1 primers and standard PCR primers. When these sequences are used as the basis of NASBA P1 primers they have the general structure X₁-SEQ, wherein X₁ represents a sequence comprising a promoter and SEQ represents the HPV-specific sequence. The promoter sequence X₁ is essential. However, when the same sequences are used as the basis of standard PCR primers it is not necessary to include X₁. The phrase “sequence number” as used in the claims is to be interpreted accordingly.

For the avoidance of doubt, the phrase “a NASBA P1 primer comprising sequence number 1” is to be interpreted as requiring the presence of an X₁ sequence 5′ to the HPV-specific sequence listed as sequence number 1, whereas the phrase “a PCR primer comprising sequence number 1” refers to any suitable PCR primer comprising the HPV-specific sequence, X₁ not being an essential feature of a PCR primer. The phrase “an oligonucleotide primer including sequence number n” is taken to encompass NASBA P1, NASBA P2 and PCR primers.

A second type of oligonucleotide provided by the invention are NASBA primer 2 oligonucleotides (also referred to herein as NASBA P2 primers) which generally comprise a sequence of approximately 20 bases substantially identical to a region of the target mRNA. The oligonucleotide sequences denoted in Table 1 as “NASBA P2/PCR” are suitable for use in both NASBA P1 primers and standard PCR primers.

Oligonucleotides intended for use as NASBA P2 primers may, in a particular but non-limiting embodiment, further comprise a sequence of nucleotides at the 5′ end which is unrelated to the target mRNA but which is capable of hybridising to a generic detection probe. The detection probe will preferably be labelled, for example with a fluorescent, luminescent or enzymatic label. In one embodiment the detection probe is labelled with a label that permits detection using ECL™ technology, although it will be appreciated that the invention is in no way limited to this particular method of detection. In a preferred embodiment the 5′ end of the primer 2 oligonucleotides may comprise the sequence GATGCAAGGTCGCATATGAG. This sequence is capable of hybridising to a generic ECL™ probe commercially available from Organon Teknika having the following structure:

Ru(bpy)₃²⁺-GAT GCA AGG TCG CAT ATG AG-3′

In a different embodiment the primer 2 oligonucleotide may incorporate “molecular beacons” technology, which is known in the art and described, for example, in WO 95/13399 by Tyagi and Kramer, Nature Biotechnology. 14: 303-308, 1996, to allow for real-time monitoring of the NASBA reaction.

A third type of oligonucleotide molecules provided by the invention are target-specific probe oligonucleotides (denoted “probe” in Table 1). The probe oligonucleotides generally comprise a sequence of approximately 20-25 bases substantially identical to a region of the target mRNA, or the complement thereof. The probe oligonucleotides may be used as target-specific hybridisation probes for detection of the products of a NASBA or PCR reaction. In this connection the probe oligonucleotides may be coupled to a solid support, such as paramagnetic beads, to form a capture probe (see below). In a preferred embodiment the 5′ end of the probe oligonucleotide may be labelled with biotin. The addition of a biotin label facilitates attachment of the probe to a solid support via a biotin/streptavidin or biotin/avidin linkage.

A fourth type of oligonucleotide molecules provided by the invention are target-specific probes incorporating “molecular beacons” technology which is known in the art and described, for example, by Tyagi and Kramer, Nature Biotechnology. 14: 303-308, 1996 and in WO 95/13399.

The term “molecular beacons probes” as used herein is taken to mean molecules having the structure:
X₂-arm₁-target-arm₂-X₃
wherein “target” represents a target-specific sequence of nucleotides, “X₂” and “X₃” represent a fluorescent moiety and a quencher moiety capable of substantially or completely quenching the fluorescence from the fluorescent moiety when the two are held together in close proximity and “arm₁” and “arm₂” represent complementary sequences capable of forming a stem duplex.

The invention provides molecular beacons probes incorporating a target-specific sequence comprising one of sequence numbers 6, 18, 35, 43, 123, 124 or 125.

Suitable pairs of arm₁ and arm₂ sequences for use with these HPV-specific sequences include, but not exclusively, the following:

For use with sequence number 6:

CGCATG---------CATGCG
CCAGCT---------AGCTGG
CACGC-----------GCGTG
CGATCG---------CGATCG

For use with sequence number 18:

CGCATG---------CATGCG
CCGTCG---------CGACGG
CGGACC---------GGTCCG
CGATCG---------CGATCG

For use with sequence number 35:

CCGAAGG-------CCTTCGG
CCGTCG---------CGACGG
CACGTCG-------CGACGTG
CGCAGC---------GCTGCG
CGATCG---------CGATCG

For use with sequence number 43:

CCAAGC---------GCTTGG
CCAAGCG-------CGCTTGG
CCCAGC---------GCTGGG
CCAAAGC-------GCTTTGG
CCTGC-----------GCAGG
CGATCG---------CGATCG

For use with sequence number 123:

CGCATG---------CATGCG
CCGTCG---------CGACGG
CCACCC----------GGGTGG
CGATCG----------CGATCG

For use with sequence number 124:

CCAAGC----------GCTTGG
CCAAGCC--------GGCTTGG
CCAAGCG--------GCGTTGG
CCAGCG----------CGCTGG
CGATCG----------CGATCG

For use with sequence number 125:

CCAAGC----------GCTTGG
CGCATG----------CATGCG
CCCAGC----------GCTGGG
CGATCG----------CGATCG

The use of probe molecules incorporating molecular beacons technology allows for real-time monitoring of amplification reactions, such as NASBA or RT-PCR reactions. The use of molecular beacons technology allows for real-time monitoring of the NASBA reaction (see Leone et al., Nucleic Acids Research., 1998, vol: 26, pp 2150-2155). The molecular beacons probes generally include complementary sequences flanking the HPV-specific sequence, represented herein by the notation arm₁ and arm₂, which are capable of hybridising to each other form a stem duplex structure. The precise sequences of arm₁ and arm₂ are not material to the invention, except for the requirement that these sequences must be capable of forming a stem duplex when the probe is not bound to a target HPV sequence.

Molecular beacons probes also include a fluorescent moiety and a quencher moiety, the fluorescent and the quencher moieties being represented herein by the notation X₂ and X₃. As will be appreciated be the skilled reader, the fluorescer and quencher moieties are selected such that the quencher moiety is capable of substantially or completely quenching the fluorescence from the fluorescent moiety when the two moieties are in close proximity, e.g. when the probe is in the hairpin “closed” conformation in the absence of the target sequence. Upon binding to the target sequence, the fluorescent and quencher moieties are held apart such that the fluorescence of the fluorescent moiety is no longer quenched.

Many examples of suitable pairs of quencher/fluorescer moieties which may be used in accordance with the invention are known in the art (see WO 95/13399, Tyagi and Kramer, ibid). A broad range of fluorophores in many different colours made be used, including for example 5-(2′-aminoethyl)aminonaphthalene-1-sulphonic acid (EDANS), fluorescein, FAM and Texas Red (see Tyagi, Bratu and Kramer, 1998, Nature Biotechnology, 16, 49-53. The use of probes labelled with different coloured fluorophores enables “multiplex” detection of two or more different probes in a single reaction vessel. A preferred quencher is 4-(4′-dimethylaminophenylazo)benzoic acid (DABCYL), a non-fluorescent chromophore, which serves as a ‘universal’ quencher for a wide range of fluorophores. The fluorescer and quencher moieties may be covalently attached to the probe in either orientation, either with the fluorescer at or near the 5′ end and the quencher at or near the 3′ end or vice versa. Protocols for the synthesis of molecular beacon probes are known in the art. A detailed protocol for synthesis is provided in a paper entitled “Molecular Beacons: Hybridization Probes for Detection of Nucleic Acids in Homogenous Solutions” by Sanjay Tyagi et al., Department of Molecular Genetics, Public Health Research Institute, 455 First Avenue, New York, N.Y. 10016, USA, which is available online via the PHRI website (at www.phri.nyu.edu or www.molecular-beacons.org).

Suitable combinations of the NASBA P1 and NASBA P2 primer oligonucleotide molecules provided by the invention may be used to drive a NASBA amplification reaction. In order to drive a NASBA amplification reaction the primer 1 and primer 2 oligonucleotides must be capable of priming synthesis of a double-stranded DNA from a target region of mRNA. For this to occur the primer 1 and primer 2′ oligonucleotides must comprise target-specific sequences which are complementary to regions of the sense and the antisense strand of the target mRNA, respectively.

In the first phase of the NASBA amplification cycle, the so-called “non-cyclic” phase, the primer 1 oligonucleotide anneals to a complementary sequence in the target mRNA and its 3′ end is extended by the action of an RNA-dependent DNA polymerase (e.g. reverse transcriptase) to form a first-strand cDNA synthesis. The RNA strand of the resulting RNA:DNA hybrid is then digested, e.g. by the action of RNaseH, to leave a single stranded DNA. The primer 2 oligonucleotide anneals to a complementary sequence towards the 3′ end of this single stranded DNA and its 3′ end is extended (by the action of reverse transcriptase), forming a double stranded DNA. RNA polymerase is then able to transcribe multiple RNA copies from the now transcriptionally active promoter sequence within the double-stranded DNA. This RNA transcript, which is antisense to the original target mRNA, can act as a template for a further round of NASBA reactions, with primer 2 annealing to the RNA and priming synthesis of the first cDNA strand and primer 1 priming synthesis of the second cDNA strand. The general principles of the NASBA reaction are well known in the art (see Compton, J. Nature. 350: 91-92).

The target-specific probe oligonucleotides described herein may also be attached to a solid support, such as magnetic microbeads, and used as “capture probes” to immobilise the product of the NASBA amplification reaction (a single stranded RNA). The target-specific “molecular beacons” probes described herein may be used for real-time monitoring of the NASBA reaction.

In a particular embodiment the invention provides the oligonucleotide listed in Table 2, these being NASBA P1 primers and NASBA P2 primers containing the sequences listed in Table 1. The NASBA P1 primers further include a T7 promoter sequence, the NASBA P2 primers include a sequence for binding of a generic detection probe (see below) and associated probe molecules for use in the detection of HPV mRNA by NASBA. The oligonucleotides listed in Table 2 are merely illustrative and it is not intended that the scope of the invention should be limited to these specific molecules.

The NASBA P2 primers (p2)in Table 2 include the sequence GATGCAAGGTCGCATATGAG at the 5′ end; the NASBA P1 primers (p1) in Table 2 include the sequence AATTCTAATACGACTCACTATAGGGAGAAGG at the 5′ end. Oligonucleotides suitable for use as probes are identified by “po”. The P2 primers generally contain HPV sequences from the postive strand, whereas the p1 primers generally contain HPV sequences from the negative strand. nt-refers to nucleotide position in the relevant HPV genomic sequence.

TABLE 2

NASBA primer and probe sequences

| | | HPV | |
|---|---|---|---|
| Primer name | Sequence | Type | nt |
| HAe6701p2 | GATGCAAGGTCGCATATGAGCCACAGGAGCGACCC | 16 | 116 |
| | AGAAAGTTA | | |
| HAe6701p1 | AATTCTAATACGACTCACTATAGGGAGAAGGACGG | 16 | 368 |
| | TTTGTTGTATTGCTGTTC | | |
| HAe6702p2 | GATGCAAGGTCGCATATGAGCCACAGGAGCGACCC | 16 | 116 |
| | AGAAA | | |
| HAe6702p1 | AATTCTAATACGACTCACTATAGGGAGAAGGGGTT | 16 | 368 |
| | TGTTGTATTGCTGTTC | | |
| HAe6702Ap1 | AATTCTAATACGACTCACTATAGGGAGAAGGTCA | 16 | 208 |
| | CGTCGCAGTAACTGT | | |
| HAe6702Bp1 | AATTCTAATACGACTCACTATAGGGAGAAGGTTG | 16 | 191 |
| | CTTGCAGTACACACA | | |
| HAe6702Cp1 | AATTCTAATACGACTCACTATAGGGAGAAGGTGC | 16 | 186 |
| | AGTACACACATTCTA | | |
| HAe6702Dp1 | AATTCTAATACGACTCACTATAGGGAGAAGGGCA | 16 | 185 |
| | GTACACACATTCTAA | | |
| H16e6702Ap2 | GATGCAAGGTCGCATATGAGACAGTTATGCACAGA | 16 | 142 |
| | GCT | | |
| H16e6702Bp2 | GATGCAAGGTCGCATATGAGATATTAGAATGTGTG | 16 | 182 |
| | TAC | | |
| H16e6702Cp2 | GATGCAAGGTCGCATATGAGTTAGAATGTGTGTAC | 16 | 185 |
| | TGC | | |
| H16e6702Dp2 | GATGCAAGGTCGCATATGAGGAATGTGTGTACTGC | 16 | 188 |
| | AAG | | |
| H16e6702Apo | ACAGTTATGCACAGAGCT | 16 | 142 |
| H16e6702Bpo | ATATTAGAATGTGTGTAC | 16 | 182 |
| H16e6702Cpo | TTAGAATGTGTGTACTGC | 16 | 185 |
| H16e6702Dpo | GAATGTGTGTACTGCAAG | 16 | 188 |
| HAe6701po | CTTTGCTTTTCGGGATTTATGC | 16 | 235 |
| HAe6702po | TATGACTTTGCTTTTCGGGA | 16 | 230 |
| HAe6702mb1 | X₂-cgcatgTATGACTTTGCTTTTCGGGAcatgcg- | 16 | 230 |
| | X₃ | | |
| HAe6702mb2 | X₂-ccagctTATGACTTTGCTTTTCGGGAagctgg- | 16 | 230 |
| | X₃ | | |
| HAe6702mb3 | X₂-cacgcTATGACTTTGCTTTTCGGGAgcgtg-X₃ | 16 | 230 |
| H16e6702mb4 | X₂-cgatcgTATGACTTTGCTTTTCGGGAcgatcg- | 16 | 230 |
| | X₃ | | |
| HAe6703p2 | GATGCAAGGTCGCATATGAGCAGAGGAGGAGGATG | 16 | 656 |
| | AAATAGTA | | |
| HAe6703p1 | AATTCTAATACGACTCACTATAGGGAGAAGGGCAC | 16 | 741 |
| | AACCGAAGCGTAGAGTCACAC | | |
| HAe6703po | TGGACAAGCAGAACCGGACAGAGC | 16 | 687 |
| HAe6704p2 | GATGCAAGGTCGCATATGAGCAGAGGAGGAGGATG | 16 | 656 |
| | AAATAGA | | |
| HAe6704p1 | AATTCTAATACGACTCACTATAGGGAGAAGGGCAC | 16 | 741 |
| | AACCGAAGCGTAGAGTCA | | |
| HAe67O4po | AGCAGAACCGGACAGAGCCCATTA | 16 | 693 |
| H18e6701p2 | GATGCAAGGTCGCATATGAGACGATGAAATAGATG | 18 | 702 |
| | GAGTT | | |
| H18e6701p1 | AATTCTAATACGACTCACTATAGGGAGAAGGCACG | 18 | 869 |
| | GACACACAAAGGACAG | | |
| H18e6701po | AGCCGAACCACAACGTCACA | 18 | 748 |
| H18e6702p2 | GATGCAAGGTCGCATATGAGGAAAACGATGAAATA | 18 | 698 |
| | GATGGAG | | |
| H18e6702p1 | AATTCTAATACGACTCACTATAGGGAGAAGGACAC | 18 | 869 |
| | CACGGACACACAAAGGACAG | | |
| H18e6702po | GAACCACAACGTCACACAATG | 18 | 752 |
| H18e6702mb1 | X₂-cgcatgGAACCACAACGTCACACAATGcatgcg- | 18 | 752 |
| | X₃ | | |
| H18e6702mb2 | X₂-ccgtcgGAACCACAACGTCACACAATGcgacgg- | 18 | 752 |
| | X₃ | | |
| H18e6702mb3 | X₂-cggaccGAACCACAACGTCACACAATGggtccg- | 18 | 752 |
| | X₃ | | |
| H18e6702mb4 | X₂-cgatcgGAACCACAACGTCACACAATGcgatcg- | 18 | 752 |
| | X₃ | | |
| H18e6703p2 | GATGCAAGGTCGCATATGAGTTCCGGTTGACCTTC | 18 | 651 |
| | TATGT | | |
| H18e6703p1 | AATTCTAATACGACTCACTATAGGGAGAAGGGGTC | 18 | 817 |
| | GTCTGCTGAGCTTTCT | | |
| H18e6704p2 | GATGCAAGGTCGCATATGAGGCAAGACATAGAAAT | 18 | 179 |
| | AACCTG | | |
| H18e6704p1 | AATTCTAATACGACTCACTATAGGGAGAAGGACCC | 18 | 379 |
| | AGTGTTAGTTAGTT | | |
| H18e6704po | TGCAAGACAGTATTGGAACT | 18 | 207 |
| H31e6701p2 | GATGCAAGGTCGCATATGAGGGAAATACCCTACGA | 31 | 164 |
| | TGAAC | | |
| H31e6701p1 | AATTCTAATACGACTCACTATAGGGAGAAGGGGAC | 31 | 423 |
| | ACAACGGTCTTTGACA | | |
| H31e6701po | ATAGGGACGACACACCACACGGAG | 31 | 268 |
| H31e6702p2 | GATGCAAGGTCGCATATGAGGGAAATACCCTACGA | 31 | 164 |
| | TGAACTA | | |
| H31e6702p1 | AATTCTAATACGACTCACTATAGGGAGAAGGCTGG | 31 | 423 |
| | ACACAACGGTCTTTGACA | | |
| H31e6702po | TAGGGACGACACACCACACGGA | 31 | 269 |
| H31e6703p2 | GATGCAAGGTCGCATATGAGACTGACCTCCACTGT | 31 | 617 |
| | TATGA | | |
| H31e6703p1 | AATTCTAATACGACTCACTATAGGGAGAAGGTATC | 31 | 766 |
| | TACTTGTGTGCTCTGT | | |
| H31e6703po | GACAAGCAGAACCGGACACATC | 31 | 687 |
| H31e6704p2 | GATGCAAGGTCGCATATGAGTGACCTCCACTGTTA | 31 | 619 |
| | TGAGCAATT | | |
| H31e6704p1 | AATTCTAATACGACTCACTATAGGGAGAAGGTGCG | 31 | 766 |
| | AATATCTACTTGTGTGCTCT GT | | |
| H31e6704po | GGACAAGCAGAACCGGACACATCCAA | 31 | 686 |
| H31e6704mb1 | X₂-ccgaaggGGACAAGCAGAACCGGACACATCC | 31 | 686 |
| | AAccttcgg-X₃ | | |
| H31e6704mb2 | X₂-ccgtcgGGACAAGCAGAACCGGACACATCCA | 31 | 686 |
| | Acgacgg-X₃ | | |
| H31e6704mb3 | X₂- | 31 | 686 |
| | cacgtcgGGACAAGCAGAACCGGACACATCCAA | | |
| | cgacgtg-X₃ | | |
| H31e6704mb4 | X₂-cgcagcGGACAAGCAGAACCGGACACATCCAA | 31 | 686 |
| | gctgcg-X₃ | | |
| H31e6704mb5 | X₂-cgatcgGGACAAGCAGAACCGGACACATCCAA | 31 | 686 |
| | cgatcg-X₃ | | |
| H31e6705p2 | GATGCAAGGTCGCATATGAGACTGACCTCCACTGT | 31 | 617 |
| | TAT | | |
| H31e6705p1 | AATTCTAATACGACTCACTATAGGGAGAAGGCACG | 31 | 809 |
| | ATTCCAAATGAGCCCAT | | |
| H33e6701p2 | GATGCAAGGTCGCATATGAGTATCCTGAACCAACT | 33 | 618 |
| | GACCTAT | | |
| H33e6701p1 | AATTCTAATACGACTCACTATAGGGAGAAGGTTGA | 33 | 763 |
| | CACATAAACGAACTG | | |
| H33e6701po | CAGATGGACAAGCACAACC | 33 | 694 |
| H33e6703p2 | GATGCAAGGTCGCATATGAGTCCTGAACCAACTGA | 33 | 620 |
| | CCTAT | | |
| H33e6703p1 | AATTCTAATACGACTCACTATAGGGAGAAGGCCCA | 33 | 807 |
| | TAAGTAGTTGCTGTAT | | |
| H33e6703po | GGACAAGCACAACCAGCCACAGC | 33 | 699 |
| H33e6703mb1 | X₂-ccaagcGGACAAGCACAACCAGCCACAGCgct | 33 | 699 |
| | tgg-X₃ | | |
| H33e6703mb2 | X₂-ccaagcgGGACAAGCACAACCAGCCACAGC | 33 | 699 |
| | cgcttgg-X₃ | | |
| H33e6703mb3 | X₂-cccagcGGACAAGCACAACCAGCCACAGCgct | 33 | 699 |
| | ggg-X₃ | | |
| H33e6703mb4 | X₂-ccaaagcGGACAAGCACAACCAGCCACAGCg | 33 | 699 |
| | ctttgg-X₃ | | |
| H33e6703mb5 | X₂-cctgcGGACAAGCACAACCAGCCACAGCgcagg- | 33 | 699 |
| | X₃ | | |
| H33e6703mb6 | X₂-cgatcgGGACAAGCACAACCAGCCACAGCcga | 33 | 699 |
| | tcg-X₃ | | |
| H33e6702p2 | GATGCAAGGTCGCATATGAGGACCTTTGTGTCCTC | 33 | 431 |
| | AAGAA | | |
| H33e6702p1 | AATTCTAATACGACTCACTATAGGGAGAAGGAGGT | 33 | 618 |
| | CAGTTGGTTCAGGATA | | |
| H33e6702po | AGAAACTGCACTGTGACGTGT | 33 | 543 |
| H35e6701p2 | GATGCAAGGTCGCATATGAGATTACAGCGGAGTGA | 35 | 217 |
| | GGTAT | | |
| H35e6701p1 | AATTCTAATACGACTCACTATAGGGAGAAGGGTCT | 35 | 442 |
| | TTGCTTTTCAACTGGA | | |
| H35e5601po | ATAGAGAAGGCCAGCCATAT | 35 | 270 |
| H35e6702p2 | GATGCAAGGTCGCATATGAGTCAGAGGAGGAGGAA | 35 | 655 |
| | GATACTA | | |
| H35e6702p1 | AATTCTAATACGACTCACTATAGGGAGAAGGGATT | 35 | 844 |
| | ATGCTCTCTGTGAACA | | |
| H35e6703p2 | GATGCAAGGTCGCATATGAGCCCGAGGCAACTGAC | 35 | 610 |
| | CTATA | | |
| H35e6703p1 | AATTCTAATACGACTCACTATAGGGAGAAGGGTCA | 35 | 770 |
| | ATGTGTGTGCTCTGTA | | |
| H35e6702po | GACAAGCAAAACCAGACACCTCCAA | 35 | 692 |
| H35e6703po | GACAAGCAAAACCAGACACC | 35 | 692 |
| H52e6701p2 | GATGCAAGGTCGCATATGAGTTGTGTGAGGTGCTG | 52 | 144 |
| | GAAGAAT | | |
| H52e6701p1 | AATTCTAATACGACTCACTATAGGGAGAAGGCCCT | 52 | 358 |
| | CTCTTCTAATGTTT | | |
| H52e6701po | GTGCCTACGCTTTTTATCTA | 52 | 296 |
| H52e6702p2 | GATGCAAGGTCGCATATGAGGTGCCTACGCTTTTT | 52 | 296 |
| | ATCTA | | |
| H52e6702p1 | AATTCTAATACGACTCACTATAGGGAGAAGGGGGG | 52 | 507 |
| | TCTCCAACACTCTGAACA | | |
| H52e6702po | TGCAAACAAGCGATTTCA | 52 | 461 |
| H58e6701p2 | GATGCAAGGTCGCATATGAGTCAGGCGTTGGAGAC | 58 | 157 |
| | ATC | | |
| H58e6701p1 | AATTCTAATACGACTCACTATAGGGAGAAGGAGCA | 58 | 301 |
| | ATCGTAAGCACACT | | |
| H58e6702p2 | GATGCAAGGTCGCATATGAGTCTGTGCATGAAATC | 58 | 173 |
| | GAA | | |
| H58e6702p1 | AATTCTAATACGACTCACTATAGGGAGAAGGAGCA | 58 | 291 |
| | CACTTTACATACTG | | |
| H58e6701po | TGAAATGCGTTGAATGCA | 58 | 192 |
| H58e6702po | TTGCAGCGATCTGAGGTATATG | 58 | 218 |
| HBe6701p2 | GATGCAAGGTCGCATATGAGTACACTGCTGGACAA | B(11) | 514 |
| | CAT | | |
| HBe6701p1 | AATTCTAATACGACTCACTATAGGGAGAAGGTCAT | B(11) | 619 |
| | CTTCTGAGCTGTCT | | |
| HBe6702p2 | GATGCAAGGTCGCATATGAGTACACTGCTGGACAA | B(11) | 514 |
| | CATGCA | | |
| HBe6702p1 | AATTCTAATACGACTCACTATAGGGAGAAGGGTCA | B(11) | 693 |
| | CATCCACAGCAACAGGTCA | | |
| HBe6701po | GTAGGGTTACATTGCTATGA | B(11) | 590 |
| HBe6702po | GTAGGGTTACATTGCTATGAGC | B(11) | 590 |
| HBe6703p2 | GATGCAAGGTCGCATATGAGTGACCTGTTGCTGTG | B(11) | 693 |
| | GATGTGA | | |
| HBe6703p1 | AATTCTAATACGACTCACTATAGGGAGAAGGTACC | B(11) | 832 |
| | TGAATCGTCCGCCAT | | |
| HBe6703po | ATWGTGTGTCCCATCTGC | B(11) | 794 |
| HCe6701p2 | GATGCAAGGTCGCATATGAGCATGCCATAAATGTA | C(18 | 295 |
| | TAGA | 39 45) | |
| HCe6701p1 | AATTCTAATACGACTCACTATAGGGAGAAGGCACC | C(18 | 408 |
| | GCAGGCACCTTATTAA | 39 45 | |
| HCe6701po | AGAATTAGAGAATTAAGA | C(18 | 324 |
| | | 39 45 | |
| H39e6701p2 | GATGCAAGGTCGCATATGAGGCAGACGACCACTAC | 39 | 210 |
| | AGCAAA | | |
| H39e6701p1 | AATTCTAATACGACTCACTATAGGGAGAAGGACAC | 39 | 344 |
| | CGAGTCCGAGTAATA | | |
| H39e6701po | ATAGGGACGGGGAACCACT | 39 | 273 |
| H39e6702p2 | GATGCAAGGTCGCATATGAGTATTACTCGGACTCG | 39 | 344 |
| | GTGT | | |
| H39e6702p1 | AATTCTAATACGACTCACTATAGGGAGAAGGCTTG | 39 | 558 |
| | GGTTTCTCTTCGTGTTA | | |
| H39e6702po | GGACCACAAAACGGGAGGAC | 39 | 531 |
| H39e6703p2 | GATGCAAGGTCGCATATGAGGAAATAGATGAACCC | 39 | 703 |
| | GACCA | | |
| H39e6703p1 | AATTCTAATACGACTCACTATAGGGAGAAGGGCAC | 39 | 886 |
| | ACCACGGACACACAAA | | |
| H39e6703po | TAGCCAGACGGGATGAACCACAGC | 39 | 749 |
| H45e6701p2 | GATGCAAGGTCGCATATGAGAACCATTGAACCCAG | 45 | 430 |
| | CAGAAA | | |
| H45e6701p1 | AATTCTAATACGACTCACTATAGGGAGAAGGTCTT | 45 | 527 |
| | TCTTGCCGTGCCTGGTCA | | |
| H45e6702p2 | GATGCAAGGTCGCATATGAGGAAACCATTGAACCC | 45 | 428 |
| | AGCAGAAAA | | |
| H45e6702p1 | AATTCTAATACGACTCACTATAGGGAGAAGGTTGC | 45 | 558 |
| | TATACTTGTGTTTCCCTACG | | |
| H45e6701po | GTACCGAGGGCAGTGTAATA | 45 | 500 |
| H45e6702po | GGACAAACGAAGATTTCACA | 45 | 467 |
| H45e6703p2 | GATGCAAGGTCGCATATGAGGTTGACCTGTTGTGT | 45 | 656 |
| | TACCAGCAAT | | |
| H45e6703p1 | AATTCTAATACGACTCACTATAGGGAGAAGGCACC | 45 | 868 |
| | ACGGACACACAAAGGACAAG | | |
| H45e6704p2 | GATGCAAGGTCGCATATGAGCTGTTGACCTGTTGT | 45 | 654 |
| | GTTACGA | | |
| H45e6704p1 | AATTCTAATACGACTCACTATAGGGAGAAGGCCAC | 45 | 868 |
| | GGACACACAAAGGACAAG | | |
| H45e6705p2 | GATGCAAGGTCGCATATGAGGTTGACCTGTTGTGT | 45 | 656 |
| | TAGGA | | |
| H45e6705p1 | AATTCTAATACGACTCACTATAGGGAGAAGGACGG | 45 | 868 |
| | ACACACAAAGGACAAG | | |
| H45e6703po | GAGTCAGAGGAGGAAAACGATG | 45 | 686 |
| H45e6704po | AGGAAAACGATGAAGCAGATGGAGT | 45 | 696 |
| H45e6705po | ACAACTACCAGCCCGACGAGCCGAA | 45 | 730 |
| H51e6701p2 | GATGCAAGGTCGCATATGAGGGAGGAGGATGAAGT | 51 | 658 |
| | AGATA | | |
| H51e6701p1 | AATTCTAATACGACTCACTATAGGGAGAAGGGCCC | 51 | 807 |
| | ATTAACATCTGCTGTA | | |
| H51e6702p2 | GATGCAAGGTCGCATATGAGAGAGGAGGAGGATGA | 51 | 655 |
| | AGTAGATA | | |
| H51e6702p1 | AATTCTAATACGACTCACTATAGGGAGAAGGACGG | 51 | 829 |
| | GCAAACCAGGCTTAGT | | |
| H51e6701po | GCAGGTGTTCAAGTGTAGTA | 51 | 747 |
| H51e6702po | TGGCAGTGGAAAGCAGTGGAGACA | 51 | 771 |
| H56e6701p2 | GATGCAAGGTCGCATATGAGTTGGGGTGCTGGAGA | 56 | 519 |
| | CAAACATCT | | |
| H56e6701p1 | AATTCTAATACGACTCACTATAGGGAGAAGGTTCA | 56 | 665 |
| | TCCTCATCCTCATCCTCTGA | | |
| H56e6702p2 | GATGCAAGGTCGCATATGAGTGGGGTGCTGGAGAC | 56 | 520 |
| | AAACATC | | |
| H56e6702p1 | AATTCTAATACGACTCACTATAGGGAGAAGGCATC | 56 | 665 |
| | CTCATCCTCATCCTCTGA | | |
| H56e6703p2 | GATGCAAGGTCGCATATGAGTTGGGGTGCTGGAGA | 56 | 519 |
| | CAAACAT | | |
| H56e6703p1 | AATTCTAATACGACTCACTATAGGGAGAAGGCCAC | 56 | 764 |
| | AAACTTACACTCACAACA | | |
| H56e6701po | AAAGTACCAACGCTGCAAGACGT | 56 | 581 |
| H56e6702po | AGAACTAACACCTCAAACAGAAAT | 56 | 610 |
| H56e6703po | AGTACCAACGCTGCAAGACGTT | 56 | 583 |
| H56e6703po1 | TTGGACAGCTCAGAGGATGAGG | 56 | 656 |
| H56e6704p2 | GATGCAAGGTCGCATATGAGGATTTTCCTTATGCA | 56 | 279 |
| | GTGTG | | |
| H56e6704p1 | AATTCTAATACGACTCACTATAGGGAGAAGGGACA | 56 | 410 |
| | TCTGTAGCACCTTATT | | |
| H56e6704po | GACTATTCAGTGTATGGAGC | 56 | 348 |
| HPVAPO1A | CAACTGAYCTMYACTGTTATGA | A(16 | |
| | | 31 35) | |
| HPVApo1Amb1 | X₂-cgcatgCAACTGAYCTMYACTGTTATGAcatgcg- | A(16 | |
| | X₃ | 31 35) | |
| HPVApo1Amb2 | X₂-ccgtcgCAACTGAYCTMYACTGTTATGAcga | A(16 | |
| | cgg-X₃ | 31 35) | |
| HPVApo1Amb3 | X₂-ccacccCAACTGAYCTMYACTGTTATGAgg | A(16 | |
| | gtgg-X₃ | 31 35) | |
| HPVApo1Amb4 | X₂-cgatcgCAACTGAYCTMYACTGTTATGAcga | A(16 | |
| | tcg-X₃ | 31 35) | |
| HPVAPO4A | GAAMCAACTGACCTAYWCTGCTAT | A(33 | |
| | | 52 58) | |
| HPVAPO4Amb1 | X₂-ccaagcGAAMCAACTGACCTAYWCTGCTATgc | A(33 | |
| | ttgg-X₃ | 52 58) | |
| HPVAPO4Amb2 | X₂-ccaagccGAAMCAACTGACCTAYWCTGCTAT | A(33 | |
| | ggcttgg-X₃ | 52 58) | |
| HPVAPO4Amb3 | X₂-ccaagcgGAAMCAACTGACCTAYWCTGCTA | A(33 | |
| | Tcgcttgg-X₃ | 52 58) | |
| HPVAPO4Amb4 | X₂-ccagcgGAAMCAACTGACCTAYWCTGCTATcg | A(33 | |
| | ctgg-X₃ | 52 58) | |
| HPVAPO4Amb5 | X₂-cgatcgGAAMCAACTGACCTAYWCTGCTATcg | A(33 | |
| | atcg-X₃ | 52 58) | |
| HPVCPO4 | AAGACATTATTCAGACTC | C(18 | |
| | | 45 39) | |
| HPVCPO4Amb1 | X₂-ccaagcAAGACATTATTCAGACTCgcttgg-X₃ | C(18 | |
| | | 45 39) | |
| HPVCPO4Amb2 | X₂-cgcatgAAGACATTATTCAGACTCcatgcg-X₃ | C(18 | |
| | | 45 39) | |
| HPVCPO4Amb3 | X₂-cccagcAAGACATTATTCAGACTCgctggg-X₃ | C(18 | |
| | | 45 39) | |
| HPVCPO4Amb4 | X₂-cgatcgAAGACATTATTCAGACTCcgatcg-X₃ | C(18 | |
| | | 45 39) | |

The meaning of X₂- and -X₃ is defined above, in the discussion of “molecular beacons” probe molecules.

In a further embodiment the invention provides the oligonucleotides listed in Table 3, these being PCR primers for use in the detection of HPV mRNA by RT-PCR. These oligonucleotides are merely illustrative and it is not intended that the scope of the invention should be limited to these specific molecules:

| | | HPV | |
|---|---|---|---|
| Primer name | Sequence | type | nt |
| HAe6701PCR2 | CCACAGGAGCGACCCAGAAAGTTA | 16 | 116 |
| HAe6701PCR1 | ACGGTTTGTTGTATTGCTGTTC | 16 | 368 |
| HAe6702PCR2 | CCACAGGAGCGACCCAGAAA | 16 | 116 |
| HAe6702PCR1 | GGTTTGTTGTATTGCTGTTC | 16 | 368 |
| HAe6703PCR2 | CAGAGGAGGAGGATGAAATAGTA | 16 | 656 |
| HAe6703PCR1 | GCACAACCGAAGCTGTAGAGTCACAC | 16 | 741 |
| HAe6704PCR2 | CAGAGGAGGAGGATGAAATAGA | 16 | 656 |
| HAe6704PCR1 | GCACAACCGAAGCGTAGAGTCA | 16 | 741 |
| H18e6701PCR2 | ACGATGAAATAGATGGAGTT | 18 | 702 |
| H18e6701PCR1 | CACGGACACACAAAGGACAG | 18 | 869 |
| H18e6702PCR2 | GAAAACGATGAAATAGATGGAG | 18 | 698 |
| H18e6702PCR1 | ACACCACGGACACACAAAGGACAG | 18 | 869 |
| H18e6703PCR2 | TTCCGGTTGACCTTCTATGT | 18 | 651 |
| H18e6703PCR1 | GGTCGTCTGCTGAGCTTTCT | 18 | 817 |
| H18e6704PCR2 | GCAAGACATAGAAATAACCTG | 18 | 179 |
| H18e6704PCR1 | ACCCAGTGTTAGTTAGTT | 18 | 379 |
| H31e6701PCR2 | GGAAATACCCTACGATGAAC | 31 | 164 |
| H31e6701PCR1 | GGACACAACGGTCTTTGACA | 31 | 423 |
| H31e6702PCR2 | GGAAATACCCTACGATGAACTA | 31 | 164 |
| H31e6702PCR1 | CTGGACACAACGGTCTTTGACA | 31 | 423 |
| H31e6703PCR2 | ACTGACCTCCACTGTTATGA | 31 | 617 |
| H31e6703PCR1 | TATCTACTTGTGTGCTCTGT | 31 | 766 |
| H31e6704PCR2 | TGACCTCCACTGTTATGAGCAATT | 31 | 619 |
| H31e6704PCR1 | TGCGAATATCTACTTGTGTGCTCT GT | 31 | 766 |
| H31e6705PCR2 | ACTGACCTCCACTGTTAT | 31 | 617 |
| H31e6705PCR1 | CACGATTCCAAATGAGCCCAT | 31 | 809 |
| H33e6701PCR2 | TATCCTGAACCAACTGACCTAT | 33 | 618 |
| H33e6701PCR1 | TTGACACATAAACGAACTG | 33 | 763 |
| H33e6703PCR2 | TCCTGAACCAACTGACCTAT | 33 | 620 |
| H33e6703PCR1 | CCCATAAGTAGTTGCTGTAT | 33 | 807 |
| H33e6702PCR2 | GACCTTTGTGTCCTCAAGAA | 33 | 431 |
| H33e6702PCR1 | AGGTCAGTTGGTTCAGGATA | 33 | 618 |
| H35e6701PCR2 | ATTACAGCGGAGTGAGGTAT | 35 | 217 |
| H35e6701PCR1 | GTCTTTGCTTTTCAACTGGA | 35 | 442 |
| H35e6702PCR2 | TCAGAGGAGGAGGAAGATACTA | 35 | 655 |
| H35e6702PCR1 | GATTATGCTCTCTGTGAACA | 35 | 844 |
| H35e6703PCR2 | CCCGAGGCAACTGACCTATA | 35 | 610 |
| H35e6703PCR1 | GTCAATGTGTGTGCTCTGTA | 35 | 770 |
| H52e6701PCR2 | TTGTGTGAGGTGCTGGAAGAAT | 52 | 144 |
| H52e6701PCR1 | CCCTCTCTTCTAATGTTT | 52 | 358 |
| H52e6702PCR2 | GTGCCTACGCTTTTTATCTA | 52 | 296 |
| H52e6702PCR1 | GGGGTCTCCAACACTCTGAACA | 52 | 507 |
| H58e6701PCR2 | TCAGGCGTTGGAGACATC | 58 | 157 |
| H58e6701PCR1 | AGCAATCGTAAGCACACT | 58 | 301 |
| H58e6702PCR2 | TCTGTGCATGAAATCGAA | 58 | 173 |
| H58e6702PCR1 | AGCACACTTTACATACTG | 58 | 291 |
| HBe6701PCR2 | TACACTGCTGGACAACAT | B (11) | 514 |
| HBe6701PCR1 | TCATCTTCTGAGCTGTCT | B (11) | 619 |
| HBe6702PCR2 | TACACTGCTGGACAACATGCA | B (11) | 514 |
| HBe6702PCR1 | GTCACATCCACAGCAACAGGTCA | B (11) | 693 |
| HBe6703PCR2 | TGACCTGTTGCTGTGGATGTGA | B (11) | 693 |
| HBe6703PCR1 | TACCTGAATCGTCCGCCAT | B (11) | 832 |
| HCe6701PCR2 | CATGCCATAAATGTATAGA | C (18 | 295 |
| | | 39 45 | |
| HCe6701PCR1 | CACCGCAGGCACCTTATTAA | C (18 | 408 |
| | | 39 45 | |
| H39e6701PCR2 | GCAGACGACCACTACAGCAAA | 39 | 210 |
| H39e6701PCR1 | ACACCGAGTCCGAGTAATA | 39 | 344 |
| H39e6702PCR2 | TATTACTCGGACTCGGTGT | 39 | 344 |
| H39e6702PCR1 | CTTGGGTTTCTCTTCGTGTTA | 39 | 558 |
| H39e6703PCR2 | GAAATAGATGAACCCGACCA | 39 | 703 |
| H39e6703PCR1 | GCACACCACGGACACACAAA | 39 | 886 |
| H45e6701PCR2 | AACCATTGAACCCAGCAGAAA | 45 | 430 |
| H45e6701PCR1 | TCTTTCTTGCCGTGCCTGGTCA | 45 | 527 |
| H45e6702PCR2 | GAAACCATTGAACCCAGCAGAAAA | 45 | 428 |
| H45e6702PCR1 | TTGCTATACTTGTGTTTCCCTACG | 45 | 558 |
| H45e6703PCR2 | GTTGACCTGTTGTGTTACCAGCAAT | 45 | 656 |
| H45e6703PCR1 | CACCACGGACACACAAAGGACAAG | 45 | 868 |
| H45e6704PCR2 | CTGTTGACCTGTTGTGTTACGA | 45 | 654 |
| H45e6704PCR1 | CCACGGACACACAAAGGACAAG | 45 | 868 |
| H45e6705PCR2 | GTTGACCTGTTGTGTTACGA | 45 | 656 |
| H45e6705PCR1 | ACGGACACACAAAGGACAAG | 45 | 868 |
| H51e6701PCR2 | GGAGGAGGATGAAGTAGATA | 51 | 658 |
| H51e6701PCR1 | GCCCATTAACATCTGCTGTA | 51 | 807 |
| H51e6702PCR2 | AGAGGAGGAGGATGAAGTAGATA | 51 | 655 |
| H51e6702PCR1 | ACGGGCAAACCAGGCTTAGT | 51 | 829 |
| H56e6701PCR2 | TTGGGGTGCTGGAGACAAACATCT | 56 | 519 |
| H56e6701PCR1 | TTCATCCTCATCCTCATCCTCTGA | 56 | 665 |
| H56e6702PCR2 | TGGGGTGCTGGAGACAAACATC | 56 | 520 |
| H56e6702PCR1 | CATCCTCATCCTCATCCTCTGA | 56 | 665 |
| H56e6703PCR2 | TTGGGGTGCTGGAGACAAACAT | 56 | 519 |
| H56e6703PCR1 | CCACAAACTTACACTCACAACA | 56 | 764 |
| H56e6704PCR2 | GATTTTCCTTATGCAGTGTG | 56 | 279 |
| H56e6704PCR1 | GACATCTGTAGCACCTTATT | 56 | 410 |

Primer-Pairs and Primer-Probe Sets

The invention further provides primer-pairs and primer/probe sets for use in the detection of HPV E6 transcripts.

A “primer-pair” is taken to mean two primers which may be used in combination for amplification of a portion of an HPV E6 transcript, for example by NASBA or RT-PCR. The individual oligonucleotide primers making up the primer-pair may be supplied separately, e.g. in separate containers. A primer-pair may also be supplied as a homogenous mixture of the two primers, this mixture may include additional reagents required for the amplification reaction, as discussed below.

A “primer/probe set” is taken to mean a set of oligonucleotides comprising a primer-pair, as defined above, and at least one oligonucleotide probe which is suitable for use in detection of an amplification product generated by use of the primer-pair. The individual oligonucleotides making up the primer/probe set may be supplied separately, e.g. in separate containers or as a homogenous mixture.

In this context “primer” is taken to encompass primers suitable for use in PCR and primers suitable for use in NASBA.

The term “probe” may encompass any of the probe types described herein, including molecular beacons probes suitable for use in real-time NASBA (see below) and capture probes for immobilisation of NASBA amplification products.

Specific primer-pairs provided by the invention are given below, together with suitable probes which may be used in the detection of amplification products generated using the primer-pair. In preferred embodiments, the primer-pairs listed below may comprise a NASBA P1 primer and a NASBA P2 primer or two PCR primers. The most preferred specific primer combinations are listed, using the primer names given in Tables 2 and 3. However, it is not intended to limit the scope of the invention to these particular combinations:

Primer-pairs and probes for use in the detection of mRNA transcripts from the E6 gene of HPV 16:

(1) an oligonucleotide primer comprising sequence number 1 and an oligonucleotide primer comprising sequence number 2; oligonucleotide probe comprising sequence number 5.

Preferred NASBA primers: HAe6701p1 and HAe6701p2

Preferred PCR primers: HAe6701PCR1 and HAe6701PCR2

(2) an oligonucleotide primer comprising sequence number 3 and an oligonucleotide primer comprising sequence number 4; oligonucleotide probe comprising sequence number 6.

Preferred NASBA primers: HAe6702p1 and HAe6702p2

Preferred PCR primers: HAe 6702PCR1 and HAe6702PCR2

(3) an oligonucleotide primer comprising sequence number 7 and an oligonucleotide primer comprising sequence number 8; oligonucleotide probe comprising sequence number 9.

Preferred NASBA primers: HAe6703p1 and HAe6703p2

Preferred PCR primers: HAe6703PCR1 and HAe6703PCR2

(4) an oligonucleotide primer comprising sequence number 10 and an oligonucleotide primer comprising sequence number 11; oligonucleotide probe comprising sequence number 12.

Preferred NASBA primers: HAe6704p1 and HAe6704p2

Preferred PCR primers: HAe6704PCR1 and HAe6704PCR2

(5) an oligonucleotide primer comprising one of sequence numbers 126, 127, 128 or 129 and an oligonucleotide primer comprising sequence number 1 or sequence number 3.

(6) an oligonucleotide primer comprising sequence number 2 or sequence number 4 and an oligonucleotide primer comprising one of sequence numbers 130, 131, 132 or 133.

Primer-pairs for use in the detection of mRNA transcripts from the E6 gene of HPV 18:

(7) an oligonucleotide primer comprising sequence number 13 and an oligonucleotide primer comprising sequence number 14; oligonucleotide probe comprising sequence number 15.

Preferred NASBA primers: H18e6701p1 and H18e6701p2

Preferred PCR primers: H18e6701PCR1 and H18e6701PCR2

(8) an oligonucleotide primer comprising sequence number 16 and an oligonucleotide primer comprising sequence number 17; oligonucleotide probe comprising sequence number 18.

Preferred NASBA primers: H18e6702p1 and H18e6702p2

Preferred PCR primers: H18e6702PCR1 and H18e6702PCR2

(9) an oligonucleotide primer comprising sequence number 19 and an oligonucleotide primer comprising sequence number 20.

Preferred NASBA primers: H18e6703p1 and H18e6703p2

Preferred PCR primers: H1836703PCR1 and H18e6703PCR2

(10) an oligonucleotide primer comprising sequence number 21 and an oligonucleotide primer comprising sequence number 22; oligonucleotide probe comprising sequence number 23.

Preferred NASBA primers: H18e6704p1 and H18e6704p2

Preferred PCR primers: H18e6704PCR1 and H18e6704PCR2

Primer-pairs for use in the detection of mRNA transcripts from the E6 gene of HPV 31:

(11) an oligonucleotide primer comprising sequence number 24 and an oligonucleotide primer comprising sequence number 25; oligonucleotide probe comprising sequence number 26.

Preferred NASBA primers: H31e6701p1 and H31e6701p2

Preferred PCR primers: H31e6701PCR1 and H31e6701PCR2

(12) an oligonucleotide primer comprising sequence number 27 and an oligonucleotide primer comprising sequence number 28; oligonucleotide probe comprising sequence number 29.

Preferred NASBA primers: H31e6702p1 and H31e6702p2

Preferred PCR primers: H31e6702PCR1 and H3136702PCR2

(13) an oligonucleotide primer comprising sequence number 30 and an oligonucleotide primer comprising sequence number 31; oligonucleotide probe comprising sequence number 32.

Preferred NASBA primers: H31e6703p1 and H31e6703p2

Preferred PCR primers: H31e6703PCR1 and H31e6703PCR2

(14) an oligonucleotide primer comprising sequence number 33 and an oligonucleotide primer comprising sequence number 34; oligonucleotide probe comprising sequence number 35.

Preferred NASBA primers: R31e6704p1 and H31e6704p2

Preferred PCR primers: H31e6704PCR1 and H312e6704PCR2

(15) an oligonucleotide primer comprising sequence number 36 and an oligonucleotide primer comprising sequence number 37;

Preferred NASBA primers: H31e6705p1 and H31e6705p2

Preferred PCR primers: H31e6705PCR1 and H31e6705PCR2

Primer-pairs for use in the detection of mRNA transcripts from the E6 gene of HPV 33:

(16) an oligonucleotide primer comprising sequence number 38 and an oligonucleotide primer comprising sequence number 39; oligonucleotide probe comprising sequence number 40.

Preferred NASBA primers: H33e6701p1 and H33e6701p2

Preferred PCR primers: H33e6701PCR1 and H33e6701PCR2

(17) an oligonucleotide primer comprising sequence number 41 and an oligonucleotide primer comprising sequence number 42; oligonucleotide probe comprising sequence number 43.

Preferred NASBA primers: H33e6703p1 and H33e6703p2

Preferred PCR primers: H33e6703PCR1 and H33e6703PCR2

(18) an oligonucleotide primer comprising sequence number 44 and an oligonucleotide primer comprising sequence number 45; oligonucleotide probe comprising sequence number 46.

Preferred NASBA primers: H33e6702p1 and H33e6702p2

Preferred PCR primers: H33e6702PCR1 and H33e6702PCR2

Primer-pairs for use in the detection of mRNA transcripts from the E6 gene of HPV 35:

(19) an oligonucleotide primer comprising sequence number 47 and an oliqonucleotide primer comprising sequence number 48; oligonucleotide probe comprising sequence number 53.

Preferred NASBA primers: H35e6701p1 and H35e6701p2

Preferred PCR primers: H35e6701PCR1 and H35e6701PCR2

(20) an oligonucleotide primer comprising sequence number 49 and an oligonucleotide primer comprising sequence number 50; oligonucleotide probe comprising sequence number 54.

Preferred NASBA primers: H35e6702p1 and H35e6702p2

Preferred PCR primers: H35e6702PCR1 and H35e6702PCR2

(21) an oligonucleotide primer comprising sequence number 51 and an oligonucleotide primer comprising sequence number 52; oligonucleotide probe comprising sequence number 55.

Preferred NASBA primers: H35e6703p1 and H35e6703p2

Preferred PCR primers: H35e6703PCR1 and H35e6703PCR2

Primer-pairs for use in the detection of mRNA transcripts from the E6 gene of HPV 52:

(22) an oligonucleotide primer comprising sequence number 56 and an oligonucleotide primer comprising sequence number 57; oligonucleotide probe comprising sequence number 58.

Preferred NASBA primers: H52e6701p1 and H52e6701p2

Preferred PCR primers: H52e6701PCR1 and H52e6701PCR2

(23) an oligonucleotide primer comprising sequence number 59 and an oligonucleotide primer comprising sequence number 60; oligonucleotide probe comprising sequence number 61.

Preferred NASBA primers: H52e6702p1 and H52e6702p2

Preferred PCR primers: H52e6702PCR1 and H52e6702PCR2

Primer-pairs for use in the detection of mRNA transcripts from the E6 gene of HPV 58:

(24) an oligonucleotide primer comprising sequence number 62 and an oligonucleotide primer comprising sequence number 63; oligonucleotide probe comprising sequence number 66.

Preferred NASBA primers: H58e6701p1 and H58e6701p2

Preferred PCR primers: H58e6701PCR1 and H58e6701PCR2

(25) an oligonucleotide primer comprising sequence number 64 and an oligonucleotide primer comprising sequence number 65; oligonucleotide probe comprising sequence number 67.

Preferred NASBA primers: H58e6702p1 and H58e6702p2

Preferred PCR primers: H58e6702PCR1 and H58e6702PCR2

Primer-pairs for use in the detection of mRNA transcripts from the E6 gene of HPV 51:

(26) an oligonucleotide primer comprising sequence number 104 and an oligonucleotide primer comprising sequence number 105; oligonucleotide probe comprising sequence number 108.

Preferred NASBA primers: H51e6701p1 and H51e6701p2

Preferred PCR primers: H51e6701PCR1 and H51e6701PCR2

(27) an oligonucleotide primer comprising sequence number 106 and an oligonucleotide primer comprising sequence number 107; oligonucleotide probe comprising sequence number 109.

Preferred NASBA primers: H51e6702p1 and H51e6702p2

Preferred PCR primers: H51e6702PCR1 and H51e6702PCR2

Primer-pairs for use in the detection of mRNA transcripts from the E6 gene of HPV 56:

(28) an oligonucleotide primer comprising sequence number 110 and an oligonucleotide primer comprising sequence number 111; oligonucleotide probe comprising sequence number 116.

Preferred NASBA primers: H56e6701p1 and H56e6701p2

Preferred PCR primers: H56e6701PCR1 and H56e6701PCR2

(29) an oligonucleotide primer comprising sequence number 112 and an oligonucleotide primer comprising sequence number 113; oligonucleotide probe comprising sequence number 117.

Preferred NASBA primers: H56e6702p1 and H56e6702p2

Preferred PCR primers: H56e6702PCR1 and H56e6702PCR2

(30) an oligonucleotide primer comprising sequence number 114 and an oligonucleotide primer comprising sequence number 115; oligonucleotide probe comprising sequence number 118 or sequence number 119.

Preferred NASBA primers: H56e6703p1 and H56e6703p2

Preferred PCR primers: H56e6703PCR1 and H56e6703PCR2

(31) an oligonucleotide primer comprising sequence number 120 and an oligonucleotide primer comprising sequence number 121; oligonucleotide probe comprising sequence number 122.

Preferred NASBA primers: H56e6704p1 and H56e6704p2

Preferred PCR primers: H56e6704PCR1 and H56e6704PCR2

Primer-pairs for use in the detection of mRNA transcripts from the E6 gene of HPV 39:

(32) an oligonucleotide primer comprising sequence number 80 and an oligonucleotide primer comprising sequence number 81; oligonucleotide probe comprising sequence number 82.

Preferred NASBA primers: H39e6701p1 and H39e6701p2

Preferred PCR primers: H39e6701PCR1 and H39e6701PCR2

(33) an oligonucleotide primer comprising sequence number 83 and an oligonucleotide primer comprising sequence number 84; oligonucleotide probe comprising sequence number 85.

Preferred NASBA primers: H39e6702p1 and H39e6702p2

Preferred PCR primers: H39e6702PCR1 and H39e6702PCR2

(34) an oligonucleotide primer comprising sequence number 86 and an oligonucleotide primer comprising sequence number 87; oligonucleotide probe comprising sequence number 88.

Preferred NASBA primers: H39e6703p1 and H39e6703p2

Preferred PCR primers: H39e6703PCR1 and H39e6703PCR2

Primer-pairs for use in the detection of mRNA transcripts from the E6 gene of HPV 45:

(35) an oligonucleotide primer comprising sequence number 89 and an oligonucleotide primer comprising sequence number 90; oligonucleotide probe comprising sequence number 93.

Preferred NASBA primers: H45e6701p1 and H45e6701p2

Preferred PCR primers: H45e6701PCR1 and H45e6701PCR2

(36) an oligonucleotide primer comprising sequence number 91 and an oligonucleotide primer comprising sequence number 92; oligonucleotide probe comprising sequence number 94.

Preferred NASBA primers: H45e6702p1 and H45e6702p2

Preferred PCR primers: H45e6702PCR1 and H45e6702PCR2

(37) an oligonucleotide primer comprising sequence number 95 and an oligonucleotide primer comprising sequence number 96; oligonucleotide probe comprising sequence number 101.

Preferred NASBA primers: H45e6703p1 and H45e6703p2

Preferred PCR primers: H45e6703PCR1 and H45e6703PCR2

(38) an oligonucleotide primer comprising sequence number 97 and an oligonucleotide primer comprising sequence number 98; oligonucleotide probe comprising sequence number 102.

Preferred NASBA primers: H45e6704p1 and H45e6704p2

Preferred PCR primers: H45e6704PCR1 and H45e6704PCR2

(39) an oligonucleotide primer comprising sequence number 99 and an oligonucleotide primer comprising sequence number 100; oligonucleotide probe comprising sequence number 103.

Preferred NASBA primers: H45e6705p1 and H45e6705p2

Preferred PCR primers: H45e6705PCR1 and H45e6705PCR2

Primer-pairs for use in the detection of mRNA transcripts from the E6 gene of group B HPV:

(40) an oligonucleotide primer comprising sequence number 68 and an oligonucleotide primer comprising sequence number 69; oligonucleotide probe comprising sequence number 72.

Preferred NASBA primers: HBe6701p1 and HBe6701p2

Preferred PCR primers: HBe6701PCR1 and HBe6701PCR2

(41) an oligonucleotide primer comprising sequence number 70 and an oligonucleotide primer comprising sequence number 71; oligonucleotide probe comprising sequence number 73.

Preferred NASBA primers: HBe6702p1 and HBe6702p2

Preferred PCR primers: HBe6702PCR1 and HBe6702PCR2

(42) an oligonucleotide primer comprising sequence number 74 and an oligonucleotide primer comprising sequence number 75; oligonucleotide probe comprising sequence number 76.

Preferred NASBA primers: HBe6703p1 and HBe6703p2

Preferred PCR primers: HBe6703PCR1 and HBe6703PCR2

Primer-pair for use in the detection of mRNA transcripts from the E6 gene of group C HPV:

(43) an oligonucleotide primer comprising sequence number 77 and an oligonucleotide primer comprising sequence number 78; oligonucleotide probe comprising sequence number 79.

Preferred NASBA primers: HCe6701p1 and HCe6701p2

Preferred PCR primers: HCe6701PCR1 and HCe6701PCR2

Methods of Detecting HPV

In a further aspect the invention provides a method for detecting HPV mRNA in a test sample suspected of containing HPV which comprises performing an amplification reaction on the test sample to amplify a portion of the mRNA transcribed from the E6 gene of HPV, wherein the amplification reaction is performed using one of the primer-pairs provided by the invention, as defined above.

Preferred amplification techniques which may be used to amplify a portion of the E6 mRNA are RT-PCR or NASBA.

The “test sample suspected of containing HPV” will most commonly be a clinical sample, for example a cervical scraping in the cervical screening field. The amplification reaction will preferably be carried out on a preparation of nucleic acid isolated from the test sample. The preparation of nucleic acid must include mRNA, however it need not be a preparation of purified poly A+ mRNA and preparations of total RNA or crude preparations of total nucleic acid containing both RNA and genomic DNA are also suitable as starting material for a NASBA reaction. Essentially any technique known in the art for the isolation of a preparation of nucleic acid including mRNA may be used to isolate nucleic acid from the test sample. A preferred technique is the “Boom” isolation method described in U.S. Pat. No. 5,234,809 and EP-B-0389,063. This method, which can be used to isolate a nucleic acid preparation containing both RNA and DNA, is based on the nucleic acid binding properties of silicon dioxide particles in the presence of the chaotropic agent guanidine thiocyanate (GuSCN).

Methods for the detection of HPV in a test sample using the. NASBA technique will generally comprise the following steps:
(a) assembling a reaction medium comprising a primer-pair according to the invention, an RNA directed DNA polymerase, a ribonuclease that hydrolyses the RNA strand of an RNA-DNA hybrid without hydrolysing single or double stranded RNA or DNA, an RNA polymerase that recognises said promoter, and ribonucleoside and deoxyribonucleoside triphosphates;
(b) incubating said reaction medium with a preparation of nucleic acid isolated from a test sample suspected of containing HPV under reaction conditions which permit a NASBA amplification reaction; and
(c) detecting and/or quantitatively measuring any HPV-specific product of the NASBA amplification reaction.

Detection of the specific product(s) of the NASBA reaction (i.e. sense and/or antisense copies of the target RNA) may be carried out in a number of different ways. In one approach the NASBA product(s) may be detected with the use of an HPV-specific hybridisation probe capable of specifically annealing to the NASBA product. The hybridisation probe may be attached to a revealing label, for example a fluorescent, luminescent, radioactive or chemiluminescent compound or an enzyme label or any other type of label known to those of ordinary skill in the art. The precise nature of the label is not critical, but it should be capable of producing a signal detectable by external means, either by itself or in conjunction with one or more additional substances (e.g. the substrate for an enzyme).

Also within the scope of the invention is so-called “real-time NASBA” which allows continuous monitoring of the formation of the product of the NASBA reaction over the course of the reaction. In a preferred embodiment this may be achieved using a “molecular beacons” probe comprising an HPV-specific sequence capable of annealing to the NASBA product, a stem-duplex forming oligonucleotide sequence and a pair of fluorescer/quencher moieties, as known in the art described herein. If the molecular beacons probe is added to the reaction mixture prior to amplification it may be possible to monitor the formation of the NASBA product in real-time (Leone et al., Nucleic Acids Research, 1998, Vol 26, 2150-2155).

In a further approach, the molecular beacons technology may be incorporated into the primer 2 oligonucleotide allowing real-time monitoring of the NASBA reaction without the need for a separate hybridisation probe.

In a still further approach the products of the NASBA reaction may be monitored using a generic labelled detection probe which hybridises to a nucleotide sequence in the 5′ terminus of the primer 2 oligonucleotide. This is equivalent to the “NucliSens™” detection system supplied by Organon Teknika. In this system specificity for NASBA products derived from the target HPV mRNA may be conferred by using HPV-specific capture probes comprising probe oligonucleotides as described herein attached to a solid support such as a magnetic microbead. Most preferably the generic labelled detection probe is the ECL™ detection probe supplied by Organon Teknika. NASBA amplicons are hybridized to the HPV-specific capture probes and the generic ECL probe (via a complementary sequence on primer 2). Following hybridization the bead/amplicon/ECL probe complexes may be captured at the magnet electrode of an automatic ECL reader (e.g. the NucliSens™ reader supplied by Organon Teknika. Subsequently, a voltage pulse triggers the ECL™ reaction.

Also provided by the invention are reagent kits for use in the detection of HPV by NASBA, the kits comprising a primer-pair cocktail according to the invention. The reagent kits may further comprise a mixture of enzymes required for the NASBA reaction, specifically an enzyme mixture containing an RNA directed DNA polymerase (e.g. a reverse transcriptase), a ribonuclease that hydrolyses the RNA strand of an RNA-DNA hybrid without hydrolysing single or double stranded RNA or DNA (e.g. RNaseH) and an RNA polymerase. The RNA polymerase should be one which recognises the promoter sequence present in the 5′ terminal region of the NASBA P1 primer oligonucleotides in the oligonucleotide primer sets supplied in the reagent kit. The kit may also comprise a supply of NASBA buffer containing the ribonucleosides and deoxyribonucleosides required for RNA and DNA synthesis. The composition of a standard NASBA reaction buffer will be well known to those skilled in the art.

In certain embodiments the kit may further contain one or more capture probes, comprising a probe oligonucleotide attached to a solid support as described above, for immobilising the products of a specific NASBA reaction. The kit may still further contain labelled generic detection probes. Advantageously, the detection probes may comprise a sequence of nucleotides complementary to a non-HPV sequence present at the 5′ terminal end of the NASBA P2 primer oligonucleotides present in the reagent kit.

In still further embodiments the kit may further contain one or more molecular beacon probes according to the invention. The molecular beacon probes may be supplied as a separate reagent within the kit. Alternatively, the NASBA primers and molecular beacons probe may be supplied as a primer/probe mixture. Such a mixture including the NASBA P1 and P2 primers and also a molecular beacons probe is convenient for use in “real-time” NASBA, wherein the NASBA amplification reaction and detection of an amplification product are performed simultaneously in a single reaction vessel.

The invention will be further understood with reference to the following, non-limiting, Example:

### EXAMPLE 1

### Real-Time NASBA

Collection and Preparation of Clinical Samples

Cervical cytobrush samples are collected in 9 ml lysis buffer (5M Guanidine thiocyanate) prior to RNA/DNA extraction. Since RNA is best protected in the 5M guanidine thiocyanate at −70° C. only 1 ml of the total volume of sample is used for each extraction round. 2-3 tubes with the RNA/DNA are stored at −167° C. and the rest stored at −70° C. RNA and DNA were automatically isolated according to the “Booms” isolation method from Organon Teknika (Organon Teknika B. V., Boselind 15, P.O. Box 84, 5280 A B Baxtel, The Netherlands; now Biomërieux, 69280 Marcy l'Etoile, France).

The following procedure was carried out using reagents from the Nuclisens™ Basic Kit, supplied by Organon Teknika. Procedure for n=10 samples:

1. Prepare enzyme solution.

Add 55 μl of enzyme diluent (from Nuclisens™ Basic Kit; contains sorbitol in aqueous solution) to each of 3 lyophilized enzyme spheres (from Nuclisens™ Basic Kit; contains AMV-RT, RNase H, T7 RNA polymerase and BSA). Leave this enzyme solution at least for 20 minutes at room temperature. Gather the enzyme solutions in one tube, mix well by flicking the tube with your finger, spin down briefly and use within 1 hour. Final concentrations in the enzyme mix are 375 mM sorbitol, 2.5 μg BSA, 0.08 U RNase H, 32 U T7 RNA polymerase and 6.4 U AMV-reverse transcriptase.

2. Prepare reagent sphere/KCl solution.

For 10 samples: add 80 μl reagent sphere diluent (from Nuclisens™ Basic Kit; contains Tris/HCl (pH 8.5), 45% DMSO) to the lyophilized reagent sphere (from Nuclisens™ Basic Kit; contains nucleotides, dithiotreitol and MgCl₂) and immediately vortex well. Do this with 3 reagent spheres and mix the solutions in one tube.

Add 3 μl NASBA water (from Nuclisens™ Basic Kit) to the reconstituted reagent sphere solution and mix well.

Add 56 μl of KCl stock solution (from Nuclisens™ Basic Kit) and mix well. Use of this KCl/water mixture will result in NASBA reactions with a final KCl concentration of 70 mM. Final concentrations in the reagent/KCl solution are 1 mM of each dNTP, 2 mM of ATP, UTP and CTP, 1.5 mM GTP, and 0.5 mM ITP, 0.5 mM dithiotreitol, 70 mM KCl, 12 mM MgCl₂, 40 mM Tris-HCl (pH 8.5).

3. Prepare primer/probe solution containing target-specific primers and molecular beacon probe.

For each target reaction transfer 91 μl of the reagent sphere/KCl solution (prepared in step 2) into a fresh tube. Add 25 μl of primers/molecular beacon probe solution (to give final concentration of ˜0.1-0.5 μM each of the sense and antisense primers and ˜15-70 pmol molecular beacon probe per reaction). Mix well by vortexing. Do not centrifuge.

In case less than 10 target RNA amplifications are being performed refer to the table below for the appropriate amounts of reagent sphere solution, KCl/water solution and primers to be used. Primer solutions should be used within 30 minutes after preparation.

| | Reagent sphere | | |
|---|---|---|---|
| Reactions (n) | solution (μl) | KCl/water (μl) | Primer mix (μl) |
| 10 | 80 | 30 | 10 |
| 9 | 72 | 27 | 9 |
| 8 | 64 | 24 | 8 |
| 7 | 56 | 21 | 7 |
| 6 | 48 | 18 | 6 |
| 5 | 40 | 15 | 5 |
| 4 | 32 | 12 | 4 |
| 3 | 24 | 9 | 3 |
| 2 | 16 | 6 | 2 |
| 1 | 8 | 3 | 1 |

4. Addition of samples

For each target RNA reaction:

In a 96 well microtiter plate pipette 10 μl of the primer/probe solution (prepared in step 3) into each of 10 wells. Add 5 μl nucleic acid extract to each well. Incubate the microtiter plate for 4 minutes at 65±1° C. Cool to at 41±0.5° C. for 4 minutes. Then to each well add 5 μl enzyme solution. Immediately place the microtiter plate in a fluorescent detection instrument (e.g. NucliSens™ EasyQ Analyzer) and start the amplification.

1. An oligonucleotide molecule for use in the detection of mRNA transcribed from the E6 gene of a human papillomavirus, the oligonucleotide comprising any one of sequence numbers 1-133.

2. An oligonucleotide molecule according to claim 1 which is an oligonucleotide primer selected from:
(i) a NASBA P1 primer comprising one of sequence numbers 2, 4, 8, 11, 14, 17, 20, 22, 25, 28, 31, 34, 37, 39, 42, 45, 48, 50, 52, 57, 60, 63, 65, 69, 71, 75, 78, 81, 84, 87, 90, 92, 96, 98, 100, 105, 107, 111, 113, 115, 121, 126, 127, 128 or 129;
(ii) a NASBA P2 primer comprising one of sequence numbers 1, 3, 7, 10, 13, 16, 19, 21, 24, 27, 30, 33, 36, 38, 41, 44, 47, 49, 51, 56, 62, 64, 68, 70, 74, 77, 80, 83, 86, 89, 91, 95, 97, 99, 104, 106, 110, 112, 114, 120, 103, 131, 132 or 133; and
(iii) a PCR primer comprising one of sequence numbers 1, 3, 7, 10, 13, 16, 19, 21, 24, 27, 30, 33, 36, 38, 41, 44, 47, 49, 51, 56, 59, 62, 64, 68, 70, 74, 77, 80, 83, 86, 89, 91, 95, 97, 99, 104, 106, 110, 112, 114, 120, 2, 4, 8, 11, 14, 17, 20, 22, 25, 28, 31, 34, 37, 39, 42, 45, 48, 50, 52, 57, 60, 63, 65, 69, 71, 75, 78, 81, 84 87, 90, 92, 96, 98, 100, 105, 107, 111, 113, 115, 121, 126, 127, 128, 129, 130, 131, 132 or 133:

3. An oligonucleotide primer according to claim 2 which is a NASBA P1 primer having the sequence AATTCTAATACGACTCACTATAGGGAGAAGG-SEQ, wherein SEQ represents any one of sequence numbers 2, 4, 8, 11, 14, 17, 20, 22, 25, 28, 31, 34, 37, 39, 42, 45, 48, 50, 52, 57, 60, 63, 65, 69, 71, 75, 78, 81, 84 87, 90, 92, 96, 98, 100, 105, 107, 111, 113, 115, 121, 126, 127, 128 or 129, and wherein AATTCTAATACGACTCACTATAGGGAGAAGG is SEQ ID NO:385.

4. An oligonucleotide primer according to claim 2 which is a NASBA P2 primer having the sequence GATGCAAGGTCGCATATGAG-SEQ wherein SEQ represents any one of sequence numbers 1, 3, 7, 10, 13, 16, 19, 21, 24, 27, 30, 33, 36, 38, 41, 44, 47, 49, 51, 56, 59, 62, 64, 68, 70, 74, 77, 80, 83, 86, 89, 91, 95, 97, 99, 104, 106, 110, 112, 114, 120, 130, 131, 132 or 133, and wherein GATGCAAGGTCGCATATGAG is SEQ ID NO:387.

5. An oligonucleotide molecule according to claim 1 which is a probe for use in the detection of mRNA transcribed from the E6 gene of a human papillomavirus comprising one of sequence numbers: 5, 6, 9, 12, 15, 18, 23, 26, 29, 32, 35, 40, 43, 46, 53, 54, 55, 58, 61, 66, 67, 72, 73, 76, 82, 85, 88, 93, 94, 101, 102, 103, 108, 109, 116, 117, 118, 119, 122, 130, 131, 132 or 133.

6.-7. (canceled)

8. An oligonucleotide primer-pair for use in the detection of mRNA transcripts from the E6 gene of HPV 31, comprising:
a NASBA P2 primer comprising sequence number 30 and a NASBA P1 primer comprising sequence number 31.

9. An oligonucleotide primer-pair for use in the detection of mRNA transcripts from the E6 gene of HPV 33, comprising:
a NASBA P2 primer comprising sequence number 38 and a NASBA P1 primer comprising sequence number 39.

10-15. (canceled)

16. An oligonucleotide primer-pair for use in the detection of mRNA transcripts from the E6 gene of HPV 45, comprising:
a NASBA P2 primer comprising sequence number 89 and a NASBA P1 primer comprising sequence number 90.

17-20. (canceled)

21. A primer/probe set comprising a primer-pair according to any one of claims 8, 9 or 16 and at least one oligonucleotide probe specific for amplification products generated using the primer-pair.

22. A method of detecting HPV mRNA in a test sample suspected of containing HPV which comprises performing an a nucleic acid sequence based amplification (NASBA) reaction on a preparation of nucleic acid isolated from the test sample to amplify a portion of the mRNA transcribed from the E6 gene of HPV, wherein the amplification reaction is performed using a primer-pair according to any one of claims 8, 9 or 16.

23-24. (canceled)

25. A method according to claim 22 which comprises:
(a) assembling a reaction mixture comprising said primer-pair, an RNA directed DNA polymerase, a ribonuclease that hydrolyses the RNA strand of an RNA-DNA hybrid without hydrolysing single or double stranded RNA or DNA, an RNA polymerase that recognises said promoter, and ribonucleoside and deoxyribonucleoside triphosphates;
(b) incubating said reaction mixture with a preparation of nucleic acid isolated from a test sample suspected of containing HPV under reaction conditions which permit a NASBA amplification reaction; and
(c) detecting and/or quantitatively measuring any HPV-specific product of the NASBA amplification reaction.

26. A method according to claim 25 wherein step (c) comprises real-time detection of an HPV-specific product of the NASBA amplification reaction.

27. A method according to claim 25 wherein the reaction mixture further comprises a molecular beacons probe oligonucleotide and the formation of any HPV-specific NASBA product in the NASBA reaction is monitored by detecting fluorescence from the fluorescent moiety included in the molecular beacons probe.

28. (canceled)

29. A reagent kit for use in the detection of HPV by NASBA, the kit comprising an oligonucleotide primer-pair as defined in any one of claims 8, 9 or 16 and optionally an enzyme mixture comprising an RNA directed DNA polymerase, a ribonuclease that hydrolyses the RNA strand of an RNA-DNA hybrid without hydrolysing single or double stranded RNA or DNA, and an RNA polymerase that recognises the promoter sequence present in at least one NASBA P1 primer oligonucleotide included in the reagent kit.

30. An oligonucleotide molecule according to claim 5 which is a molecular beacon probe.

31. A method according to claim 26 wherein the reaction mixture further comprises a molecular beacons probe oligonucleotide and the formation of any HPV-specific NASBA product in the NASBA reaction is monitored by detecting fluorescence from the fluorescent moiety included in the molecular beacons probe.


SEQUENCE LISTING (387 sequences)

SEQ ID NO: 1 - DNA, 24 bp, Human papillomavirus type 16
ccacaggagcgacccagaaagtta

SEQ ID NO: 2 - DNA, 22 bp, Human papillomavirus type 16
acggtttgttgtattgctgttc

SEQ ID NO: 3 - DNA, 20 bp, Human papillomavirus type 16
ccacaggagcgacccagaaa

SEQ ID NO: 4 - DNA, 20 bp, Human papillomavirus type 16
ggtttgttgtattgctgttc

SEQ ID NO: 5 - DNA, 22 bp, Human papillomavirus type 16
ctttgcttttcgggatttatgc

SEQ ID NO: 6 - DNA, 20 bp, Human papillomavirus type 16
tatgactttgcttttcggga

SEQ ID NO: 7 - DNA, 23 bp, Human papillomavirus type 16
cagaggaggaggatgaaatagta

SEQ ID NO: 8 - DNA, 25 bp, Human papillomavirus type 16
gcacaaccgaagcgtagagtcacac

SEQ ID NO: 9 - DNA, 24 bp, Human papillomavirus type 16
tggacaagcagaaccggacagagc

SEQ ID NO: 10 - DNA, 22 bp, Human papillomavirus
cagaggaggaggatgaaataga

SEQ ID NO: 11 - DNA, 22 bp, Human papillomavirus type 16
gcacaaccgaagcgtagagtca

SEQ ID NO: 12 - DNA, 24 bp, Human papillomavirus type 16
agcagaaccggacagagcccatta

SEQ ID NO: 13 - DNA, 20 bp, Human papillomavirus type 18
acgatgaaatagatggagtt

SEQ ID NO: 14 - DNA, 20 bp, Human papillomavirus type 18
cacggacacacaaaggacag

SEQ ID NO: 15 - DNA, 20 bp, Human papillomavirus type 18
agccgaaccacaacgtcaca

SEQ ID NO: 16 - DNA, 22 bp, Human papillomavirus type 18
gaaaacgatgaaatagatggag

SEQ ID NO: 17 - DNA, 24 bp, Human papillomavirus type 18
acaccacggacacacaaaggacag

SEQ ID NO: 18 - DNA, 21 bp, Human papillomavirus type 18
gaaccacaacgtcacacaatg

SEQ ID NO: 19 - DNA, 20 bp, Human papillomavirus type 18
ttccggttgaccttctatgt

SEQ ID NO: 20 - DNA, 20 bp, Human papillomavirus type 18
ggtcgtctgctgagctttct

SEQ ID NO: 21 - DNA, 21 bp, Human papillomavirus type 18
gcaagacatagaaataacctg

SEQ ID NO: 22 - DNA, 18 bp, Human papillomavirus type 18
acccagtgttagttagtt

SEQ ID NO: 23 - DNA, 20 bp, Human papillomavirus type 18
tgcaagacagtattggaact

SEQ ID NO: 24 - DNA, 20 bp, Human papillomavirus type 31
ggaaataccctacgatgaac

SEQ ID NO: 25 - DNA, 20 bp, Human papillomavirus type 31
ggacacaacggtctttgaca

SEQ ID NO: 26 - DNA, 24 bp, Human papillomavirus type 31
atagggacgacacaccacacggag

SEQ ID NO: 27 - DNA, 22 bp, Human papillomavirus type 31
ggaaataccctacgatgaacta

SEQ ID NO: 28 - DNA, 22 bp, Human papillomavirus type 31
ctggacacaacggtctttgaca

SEQ ID NO: 29 - DNA, 22 bp, Human papillomavirus type 31
tagggacgacacaccacacgga

SEQ ID NO: 30 - DNA, 20 bp, Human papillomavirus type 31
actgacctccactgttatga

SEQ ID NO: 31 - DNA, 20 bp, Human papillomavirus type 31
tatctacttgtgtgctctgt

SEQ ID NO: 32 - DNA, 22 bp, Human papillomavirus type 31
gacaagcagaaccggacacatc

SEQ ID NO: 33 - DNA, 24 bp, Human papillomavirus type 31
tgacctccactgttatgagcaatt

SEQ ID NO: 34 - DNA, 26 bp, Human papillomavirus type 31
tgcgaatatctacttgtgtgctctgt

SEQ ID NO: 35 - DNA, 26 bp, Human papillomavirus type 31
ggacaagcagaaccggacacatccaa

SEQ ID NO: 36 - DNA, 18 bp, Human papillomavirus type 31
actgacctccactgttat

SEQ ID NO: 37 - DNA, 21 bp, Human papillomavirus type 31
cacgattccaaatgagcccat

SEQ ID NO: 38 - DNA, 22 bp, Human papillomavirus type 33
tatcctgaaccaactgacctat

SEQ ID NO: 39 - DNA, 19 bp, Human papillomavirus type 33
ttgacacataaacgaactg

SEQ ID NO: 40 - DNA, 19 bp, Human papillomavirus type 33
cagatggacaagcacaacc

SEQ ID NO: 41 - DNA, 20 bp, Human papillomavirus type 33
tcctgaaccaactgacctat

SEQ ID NO: 42 - DNA, 20 bp, Human papillomavirus type 33
cccataagtagttgctgtat

SEQ ID NO: 43 - DNA, 23 bp, Human papillomavirus type 33
ggacaagcacaaccagccacagc

SEQ ID NO: 44 - DNA, 20 bp, Human papillomavirus
gacctttgtgtcctcaagaa

SEQ ID NO: 45 - DNA, 20 bp, Human papillomavirus type 33
aggtcagttggttcaggata

SEQ ID NO: 46 - DNA, 21 bp, Human papillomavirus type 33
agaaactgcactgtgacgtgt

SEQ ID NO: 47 - DNA, 20 bp, Human papillomavirus type 35
attacagcggagtgaggtat

SEQ ID NO: 48 - DNA, 20 bp, Human papillomavirus type 35
gtctttgcttttcaactgga

SEQ ID NO: 49 - DNA, 22 bp, Human papillomavirus type 35
tcagaggaggaggaagatacta

SEQ ID NO: 50 - DNA, 20 bp, Human papillomavirus type 35
gattatgctctctgtgaaca

SEQ ID NO: 51 - DNA, 20 bp, Human papillomavirus
cccgaggcaactgacctata

SEQ ID NO: 52 - DNA, 20 bp, Human papillomavirus type 35
gtcaatgtgtgtgctctgta

SEQ ID NO: 53 - DNA, 20 bp, Human papillomavirus
atagagaaggccagccatat

SEQ ID NO: 54 - DNA, 25 bp, Human papillomavirus type 35
gacaagcaaaaccagacacctccaa

SEQ ID NO: 55 - DNA, 20 bp, Human papillomavirus type 35
gacaagcaaaaccagacacc

SEQ ID NO: 56 - DNA, 22 bp, Human papillomavirus type 52
ttgtgtgaggtgctggaagaat

SEQ ID NO: 57 - DNA, 18 bp, Human papillomavirus type 52
ccctctcttctaatgttt

SEQ ID NO: 58 - DNA, 20 bp, Human papillomavirus
gtgcctacgctttttatcta

SEQ ID NO: 59 - DNA, 20 bp, Human papillomavirus type 52
gtgcctacgctttttatcta

SEQ ID NO: 60 - DNA, 22 bp, Human papillomavirus
ggggtctccaacactctgaaca

SEQ ID NO: 61 - DNA, 18 bp, Human papillomavirus type 52
tgcaaacaagcgatttca

SEQ ID NO: 62 - DNA, 18 bp, Human papillomavirus type 58
tcaggcgttggagacatc

SEQ ID NO: 63 - DNA, 18 bp, Human papillomavirus type 58
agcaatcgtaagcacact

SEQ ID NO: 64 - DNA, 18 bp, Human papillomavirus type 58
tctgtgcatgaaatcgaa

SEQ ID NO: 65 - DNA, 18 bp, Human papillomavirus
agcacactttacatactg

SEQ ID NO: 66 - DNA, 18 bp, Human papillomavirus type 58
tgaaatgcgttgaatgca

SEQ ID NO: 67 - DNA, 22 bp, Human papillomavirus type 58
ttgcagcgatctgaggtatatg

SEQ ID NO: 68 - DNA, 18 bp, Human papillomavirus
tacactgctggacaacat

SEQ ID NO: 69 - DNA, 18 bp, Human papillomavirus
tcatcttctgagctgtct

SEQ ID NO: 70 - DNA, 21 bp, Human papillomavirus
tacactgctggacaacatgca

SEQ ID NO: 71 - DNA, 23 bp, Human papillomavirus
gtcacatccacagcaacaggtca

SEQ ID NO: 72 - DNA, 20 bp, Human papillomavirus
gtagggttacattgctatga

SEQ ID NO: 73 - DNA, 22 bp, Human papillomavirus
gtagggttacattgctatgagc

SEQ ID NO: 74 - DNA, 22 bp, Human papillomavirus
tgacctgttgctgtggatgtga

SEQ ID NO: 75 - DNA, 19 bp, Human papillomavirus
tacctgaatcgtccgccat

SEQ ID NO: 76 - DNA, 18 bp, Human papillomavirus
atwgtgtgtcccatctgc

SEQ ID NO: 77 - DNA, 19 bp, Human papillomavirus
catgccataaatgtataga

SEQ ID NO: 78 - DNA, 20 bp, Human papillomavirus
caccgcaggcaccttattaa

SEQ ID NO: 79 - DNA, 18 bp, Human papillomavirus
agaattagagaattaaga

SEQ ID NO: 80 - DNA, 21 bp, Human papillomavirus type 39
gcagacgaccactacagcaaa

SEQ ID NO: 81 - DNA, 19 bp, Human papillomavirus type 39
acaccgagtccgagtaata

SEQ ID NO: 82 - DNA, 19 bp, Human papillomavirus type 39
atagggacggggaaccact

SEQ ID NO: 83 - DNA, 19 bp, Human papillomavirus
tattactcggactcggtgt

SEQ ID NO: 84 - DNA, 21 bp, Human papillomavirus type 39
cttgggtttctcttcgtgtta

SEQ ID NO: 85 - DNA, 20 bp, Human papillomavirus type 39
ggaccacaaaacgggaggac

SEQ ID NO: 86 - DNA, 20 bp, Human papillomavirus type 39
gaaatagatgaacccgacca

SEQ ID NO: 87 - DNA, 20 bp, Human papillomavirus
gcacaccacggacacacaaa

SEQ ID NO: 88 - DNA, 24 bp, Human papillomavirus type 39
tagccagacgggatgaaccacagc

SEQ ID NO: 89 - DNA, 21 bp, Human papillomavirus type 45
aaccattgaacccagcagaaa

SEQ ID NO: 90 - DNA, 22 bp, Human papillomavirus type 45
tctttcttgccgtgcctggtca

SEQ ID NO: 91 - DNA, 24 bp, Human papillomavirus type 45
gaaaccattgaacccagcagaaaa

SEQ ID NO: 92 - DNA, 24 bp, Human papillomavirus type 45
ttgctatacttgtgtttccctacg

SEQ ID NO: 93 - DNA, 20 bp, Human papillomavirus type 45
gtaccgagggcagtgtaata

SEQ ID NO: 94 - DNA, 20 bp, Human papillomavirus type 45
ggacaaacgaagatttcaca

SEQ ID NO: 95 - DNA, 25 bp, Human papillomavirus type 45
gttgacctgttgtgttaccagcaat

SEQ ID NO: 96 - DNA, 24 bp, Human papillomavirus type 45
caccacggacacacaaaggacaag

SEQ ID NO: 97 - DNA, 22 bp, Human papillomavirus type 45
ctgttgacctgttgtgttacga

SEQ ID NO: 98 - DNA, 22 bp, Human papillomavirus type 45
ccacggacacacaaaggacaag

SEQ ID NO: 99 - DNA, 20 bp, Human papillomavirus type 45
gttgacctgttgtgttacga

SEQ ID NO: 100 - DNA, 20 bp, Human papillomavirus type 45
acggacacacaaaggacaag

SEQ ID NO: 101 - DNA, 22 bp, Human papillomavirus type 45
gagtcagaggaggaaaacgatg

SEQ ID NO: 102 - DNA, 25 bp, Human papillomavirus type 45
aggaaaacgatgaagcagatggagt

SEQ ID NO: 103 - DNA, 25 bp, Human papillomavirus type 45
acaactaccagcccgacgagccgaa

SEQ ID NO: 104 - DNA, 20 bp, Human papillomavirus type 51
ggaggaggatgaagtagata

SEQ ID NO: 105 - DNA, 20 bp, Human papillomavirus type 51
gcccattaacatctgctgta

SEQ ID NO: 106 - DNA, 23 bp, Human papillomavirus type 51
agaggaggaggatgaagtagata

SEQ ID NO: 107 - DNA, 20 bp, Human papillomavirus type 51
acgggcaaaccaggcttagt

SEQ ID NO: 108 - DNA, 20 bp, Human papillomavirus type 51
gcaggtgttcaagtgtagta

SEQ ID NO: 109 - DNA, 24 bp, Human papillomavirus type 51
tggcagtggaaagcagtggagaca

SEQ ID NO: 110 - DNA, 24 bp, Human papillomavirus type 56
ttggggtgctggagacaaacatct

SEQ ID NO: 111 - DNA, 24 bp, Human papillomavirus type 56
ttcatcctcatcctcatcctctga

SEQ ID NO: 112 - DNA, 22 bp, Human papillomavirus type 56
tggggtgctggagacaaacatc

SEQ ID NO: 113 - DNA, 22 bp, Human papillomavirus type 56
catcctcatcctcatcctctga

SEQ ID NO: 114 - DNA, 22 bp, Human papillomavirus type 56
ttggggtgctggagacaaacat

SEQ ID NO: 115 - DNA, 22 bp, Human papillomavirus type 56
ccacaaacttacactcacaaca

SEQ ID NO: 116 - DNA, 23 bp, Human papillomavirus type 56
aaagtaccaacgctgcaagacgt

SEQ ID NO: 117 - DNA, 24 bp, Human papillomavirus type 56
agaactaacacctcaaacagaaat

SEQ ID NO: 118 - DNA, 22 bp, Human papillomavirus type 56
agtaccaacgctgcaagacgtt

SEQ ID NO: 119 - DNA, 22 bp, Human papillomavirus type 56
ttggacagctcagaggatgagg

SEQ ID NO: 120 - DNA, 20 bp, Human papillomavirus type 56
gattttccttatgcagtgtg

SEQ ID NO: 121 - DNA, 20 bp, Human papillomavirus type 56
gacatctgtagcaccttatt

SEQ ID NO: 122 - DNA, 20 bp, Human papillomavirus
gactattcagtgtatggagc

SEQ ID NO: 123 - DNA, 22 bp, Human papillomavirus type 56
caactgayctmyactgttatga

SEQ ID NO: 124 - DNA, 24 bp, Human papillomavirus
gaamcaactgacctaywctgctat

SEQ ID NO: 125 - DNA, 18 bp, Human papillomavirus
aagacattattcagactc

SEQ ID NO: 126 - DNA, 18 bp, Human papillomavirus type 16
tcacgtcgcagtaactgt

SEQ ID NO: 127 - DNA, 18 bp, Human papillomavirus type 16
ttgcttgcagtacacaca

SEQ ID NO: 128 - DNA, 18 bp, Human papillomavirus type 16
tgcagtacacacattcta

SEQ ID NO: 129 - DNA, 18 bp, Human papillomavirus type 16
gcagtacacacattctaa

SEQ ID NO: 130 - DNA, 18 bp, Human papillomavirus type 16
acagttatgcacagagct

SEQ ID NO: 131 - DNA, 18 bp, Human papillomavirus type 16
atattagaatgtgtgtac

SEQ ID NO: 132 - DNA, 18 bp, Human papillomavirus type 16
ttagaatgtgtgtactgc

SEQ ID NO: 133 - DNA, 17 bp, Human papillomavirus type 16
aatgtgtgtactgcaag

SEQ ID NO: 134 - DNA, 44 bp, Human papillomavirus
gatgcaaggtcgcatatgagccacaggagcgacccagaaagtta

SEQ ID NO: 135 - DNA, 53 bp, Human papillomavirus
aattctaatacgactcactatagggagaaggacggtttgttgtattgctgttc

SEQ ID NO: 136 - DNA, 40 bp, Human papillomavirus type 16
gatgcaaggtcgcatatgagccacaggagcgacccagaaa

SEQ ID NO: 137 - DNA, 51 bp, Human papillomavirus type 16
aattctaatacgactcactatagggagaaggggtttgttgtattgctgttc

SEQ ID NO: 138 - DNA, 49 bp, Human papillomavirus type 16
aattctaatacgactcactatagggagaaggtcacgtcgcagtaactgt

SEQ ID NO: 139 - DNA, 49 bp, Human papillomavirus type 16
aattctaatacgactcactatagggagaaggttgcttgcagtacacaca

SEQ ID NO: 140 - DNA, 49 bp, Human papillomavirus type 16
aattctaatacgactcactatagggagaaggtgcagtacacacattcta

SEQ ID NO: 141 - DNA, 49 bp, Human papillomavirus type 16
aattctaatacgactcactatagggagaagggcagtacacacattctaa

SEQ ID NO: 142 - DNA, 38 bp, Human papillomavirus type 16
gatgcaaggtcgcatatgagacagttatgcacagagct

SEQ ID NO: 143 - DNA, 38 bp, Human papillomavirus type 16
gatgcaaggtcgcatatgagatattagaatgtgtgtac

SEQ ID NO: 144 - DNA, 38 bp, Human papillomavirus type 16
gatgcaaggtcgcatatgagttagaatgtgtgtactgc

SEQ ID NO: 145 - DNA, 38 bp, Human papillomavirus type 16
gatgcaaggtcgcatatgaggaatgtgtgtactgcaag

SEQ ID NO: 146 - DNA, 18 bp, Human papillomavirus type 16
acagttatgcacagagct

SEQ ID NO: 147 - DNA, 18 bp, Human papillomavirus type 16
atattagaatgtgtgtac

SEQ ID NO: 148 - DNA, 18 bp, Human papillomavirus type 16
ttagaatgtgtgtactgc

SEQ ID NO: 149 - DNA, 18 bp, Human papillomavirus type 16
gaatgtgtgtactgcaag

SEQ ID NO: 150 - DNA, 22 bp, Human papillomavirus type 16
ctttgcttttcgggatttatgc

SEQ ID NO: 151 - DNA, 20 bp, Human papillomavirus type 16
tatgactttgcttttcggga

SEQ ID NO: 152 - DNA, 32 bp, Human papillomavirus type 16
cgcatgtatgactttgcttttcgggacatgcg

SEQ ID NO: 153 - DNA, 32 bp, Human papillomavirus type 16
ccagcttatgactttgcttttcgggaagctgg

SEQ ID NO: 154 - DNA, 30 bp, Human papillomavirus type 16
cacgctatgactttgcttttcgggagcgtg

SEQ ID NO: 155 - DNA, 32 bp, Human papillomavirus type 16
cgatcgtatgactttgcttttcgggacgatcg

SEQ ID NO: 156 - DNA, 43 bp, Human papillomavirus type 16
gatgcaaggtcgcatatgagcagaggaggaggatgaaatagta

SEQ ID NO: 157 - DNA, 56 bp, Human papillomavirus type 16
aattctaatacgactcactatagggagaagggcacaaccgaagcgtagagtcacac

SEQ ID NO: 158 - DNA, 24 bp, Human papillomavirus type 16
tggacaagcagaaccggacagagc

SEQ ID NO: 159 - DNA, 42 bp, Human papillomavirus type 16
gatgcaaggtcgcatatgagcagaggaggaggatgaaataga

SEQ ID NO: 160 - DNA, 53 bp, Human papillomavirus type 16
aattctaatacgactcactatagggagaagggcacaaccgaagcgtagagtca

SEQ ID NO: 161 - DNA, 24 bp, Human papillomavirus type 16
agcagaaccggacagagcccatta

SEQ ID NO: 162 - DNA, 40 bp, Human papillomavirus type 18
gatgcaaggtcgcatatgagacgatgaaatagatggagtt

SEQ ID NO: 163 - DNA, 51 bp, Human papillomavirus type 18
aattctaatacgactcactatagggagaaggcacggacacacaaaggacag

SEQ ID NO: 164 - DNA, 20 bp, Human papillomavirus type 18
agccgaaccacaacgtcaca

SEQ ID NO: 165 - DNA, 42 bp, Human papillomavirus type 18
gatgcaaggtcgcatatgaggaaaacgatgaaatagatggag

SEQ ID NO: 166 - DNA, 55 bp, Human papillomavirus type 18
aattctaatacgactcactatagggagaaggacaccacggacacacaaaggacag

SEQ ID NO: 167 - DNA, 21 bp, Human papillomavirus type 18
gaaccacaacgtcacacaatg

SEQ ID NO: 168 - DNA, 33 bp, Human papillomavirus type 18
cgcatggaaccacaacgtcacacaatgcatgcg

SEQ ID NO: 169 - DNA, 33 bp, Human papillomavirus type 18
ccgtcggaaccacaacgtcacacaatgcgacgg

SEQ ID NO: 170 - DNA, 33 bp, Human papillomavirus type 18
cggaccgaaccacaacgtcacacaatgggtccg

SEQ ID NO: 171 - DNA, 33 bp, Human papillomavirus type 18
cgatcggaaccacaacgtcacacaatgcgatcg

SEQ ID NO: 172 - DNA, 40 bp, Human papillomavirus type 18
gatgcaaggtcgcatatgagttccggttgaccttctatgt

SEQ ID NO: 173 - DNA, 51 bp, Human papillomavirus type 18
aattctaatacgactcactatagggagaaggggtcgtctgctgagctttct

SEQ ID NO: 174 - DNA, 41 bp, Human papillomavirus type 18
gatgcaaggtcgcatatgaggcaagacatagaaataacctg

SEQ ID NO: 175 - DNA, 49 bp, Human papillomavirus type 18
aattctaatacgactcactatagggagaaggacccagtgttagttagtt

SEQ ID NO: 176 - DNA, 20 bp, Human papillomavirus type 18
tgcaagacagtattggaact

SEQ ID NO: 177 - DNA, 40 bp, Human papillomavirus type 31
gatgcaaggtcgcatatgagggaaataccctacgatgaac

SEQ ID NO: 178 - DNA, 51 bp, Human papillomavirus type 31
aattctaatacgactcactatagggagaaggggacacaacggtctttgaca

SEQ ID NO: 179 - DNA, 24 bp, Human papillomavirus type 31
atagggacgacacaccacacggag

SEQ ID NO: 180 - DNA, 42 bp, Human papillomavirus type 31
gatgcaaggtcgcatatgagggaaataccctacgatgaacta

SEQ ID NO: 181 - DNA, 53 bp, Human papillomavirus type 31
aattctaatacgactcactatagggagaaggctggacacaacggtctttgaca

SEQ ID NO: 182 - DNA, 22 bp, Human papillomavirus type 31
tagggacgacacaccacacgga

SEQ ID NO: 183 - DNA, 40 bp, Human papillomavirus type 31
gatgcaaggtcgcatatgagactgacctccactgttatga

SEQ ID NO: 184 - DNA, 51 bp, Human papillomavirus type 31
aattctaatacgactcactatagggagaaggtatctacttgtgtgctctgt

SEQ ID NO: 185 - DNA, 22 bp, Human papillomavirus type 31
gacaagcagaaccggacacatc

SEQ ID NO: 186 - DNA, 44 bp, Human papillomavirus type 31
gatgcaaggtcgcatatgagtgacctccactgttatgagcaatt

SEQ ID NO: 187 - DNA, 57 bp, Human papillomavirus type 31
aattctaatacgactcactatagggagaaggtgcgaatatctacttgtgtgctctgt

SEQ ID NO: 188 - DNA, 26 bp, Human papillomavirus type 31
ggacaagcagaaccggacacatccaa

SEQ ID NO: 189 - DNA, 40 bp, Human papillomavirus type 31
ccgaaggggacaagcagaaccggacacatccaaccttcgg

SEQ ID NO: 190 - DNA, 38 bp, Human papillomavirus type 31
ccgtcgggacaagcagaaccggacacatccaacgacgg

SEQ ID NO: 191 - DNA, 40 bp, Human papillomavirus type 31
cacgtcgggacaagcagaaccggacacatccaacgacgtg

SEQ ID NO: 192 - DNA, 38 bp, Human papillomavirus type 31
cgcagcggacaagcagaaccggacacatccaagctgcg

SEQ ID NO: 193 - DNA, 38 bp, Human papillomavirus type 31
cgatcgggacaagcagaaccggacacatccaacgatcg

SEQ ID NO: 194 - DNA, 38 bp, Human papillomavirus type 31
gatgcaaggtcgcatatgagactgacctccactgttat

SEQ ID NO: 195 - DNA, 52 bp, Human papillomavirus type 31
aattctaatacgactcactatagggagaaggcacgattccaaatgagcccat

SEQ ID NO: 196 - DNA, 42 bp, Human papillomavirus type 31
gatgcaaggtcgcatatgagtatcctgaaccaactgacctat

SEQ ID NO: 197 - DNA, 50 bp, Human papillomavirus type 33
aattctaatacgactcactatagggagaaggttgacacataaacgaactg

SEQ ID NO: 198 - DNA, 19 bp, Human papillomavirus type 33
cagatggacaagcacaacc

SEQ ID NO: 199 - DNA, 40 bp, Human papillomavirus type 33
gatgcaaggtcgcatatgagtcctgaaccaactgacctat

SEQ ID NO: 200 - DNA, 51 bp, Human papillomavirus type 33
aattctaatacgactcactatagggagaaggcccataagtagttgctgtat

SEQ ID NO: 201 - DNA, 23 bp, Human papillomavirus type 33
ggacaagcacaaccagccacagc

SEQ ID NO: 202 - DNA, 35 bp, Human papillomavirus type 33
ccaagcggacaagcacaaccagccacagcgcttgg

SEQ ID NO: 203 - DNA, 37 bp, Human papillomavirus type 33
ccaagcgggacaagcacaaccagccacagccgcttgg

SEQ ID NO: 204 - DNA, 35 bp, Human papillomavirus type 33
cccagcggacaagcacaaccagccacagcgctggg

SEQ ID NO: 205 - DNA, 37 bp, Human papillomavirus type 33
ccaaagcggacaagcacaaccagccacagcgctttgg

SEQ ID NO: 206 - DNA, 33 bp, Human papillomavirus type 33
cctgcggacaagcacaaccagccacagcgcagg

SEQ ID NO: 207 - DNA, 35 bp, Human papillomavirus type 33
cgatcgggacaagcacaaccagccacagccgatcg

SEQ ID NO: 208 - DNA, 40 bp, Human papillomavirus type 33
gatgcaaggtcgcatatgaggacctttgtgtcctcaagaa

SEQ ID NO: 209 - DNA, 51 bp, Human papillomavirus type 33
aattctaatacgactcactatagggagaaggaggtcagttggttcaggata

SEQ ID NO: 210 - DNA, 21 bp, Human papillomavirus type 33
agaaactgcactgtgacgtgt

SEQ ID NO: 211 - DNA, 40 bp, Human papillomavirus type 35
gatgcaaggtcgcatatgagattacagcggagtgaggtat

SEQ ID NO: 212 - DNA, 51 bp, Human papillomavirus type 35
aattctaatacgactcactatagggagaagggtctttgcttttcaactgga

SEQ ID NO: 213 - DNA, 20 bp, Human papillomavirus type 35
atagagaaggccagccatat

SEQ ID NO: 214 - DNA, 42 bp, Human papillomavirus type 35
gatgcaaggtcgcatatgagtcagaggaggaggaagatacta

SEQ ID NO: 215 - DNA, 51 bp, Human papillomavirus type 35
aattctaatacgactcactatagggagaagggattatgctctctgtgaaca

SEQ ID NO: 216 - DNA, 40 bp, Human papillomavirus type 35
gatgcaaggtcgcatatgagcccgaggcaactgacctata

SEQ ID NO: 217 - DNA, 51 bp, Human papillomavirus type 35
aattctaatacgactcactatagggagaagggtcaatgtgtgtgctctgta

SEQ ID NO: 218 - DNA, 25 bp, Human papillomavirus type 35
gacaagcaaaaccagacacctccaa

SEQ ID NO: 219 - DNA, 20 bp, Human papillomavirus type 35
gacaagcaaaaccagacacc

SEQ ID NO: 220 - DNA, 42 bp, Human papillomavirus type 52
gatgcaaggtcgcatatgagttgtgtgaggtgctggaagaat

SEQ ID NO: 221 - DNA, 49 bp, Human papillomavirus type 52
aattctaatacgactcactatagggagaaggccctctcttctaatgttt

SEQ ID NO: 222 - DNA, 20 bp, Human papillomavirus type 52
gtgcctacgctttttatcta

SEQ ID NO: 223 - DNA, 40 bp, Human papillomavirus type 52
gatgcaaggtcgcatatgaggtgcctacgctttttatcta

SEQ ID NO: 224 - DNA, 53 bp, Human papillomavirus type 52
aattctaatacgactcactatagggagaaggggggtctccaacactctgaaca

SEQ ID NO: 225 - DNA, 18 bp, Human papillomavirus type 52
tgcaaacaagcgatttca

SEQ ID NO: 226 - DNA, 38 bp, Human papillomavirus type 58
gatgcaaggtcgcatatgagtcaggcgttggagacatc

SEQ ID NO: 227 - DNA, 49 bp, Human papillomavirus type 58
aattctaatacgactcactatagggagaaggagcaatcgtaagcacact

SEQ ID NO: 228 - DNA, 38 bp, Human papillomavirus type 58
gatgcaaggtcgcatatgagtctgtgcatgaaatcgaa

SEQ ID NO: 229 - DNA, 49 bp, Human papillomavirus type 58
aattctaatacgactcactatagggagaaggagcacactttacatactg

SEQ ID NO: 230 - DNA, 18 bp, Human papillomavirus type 58
tgaaatgcgttgaatgca

SEQ ID NO: 231 - DNA, 22 bp, Human papillomavirus type 58
ttgcagcgatctgaggtatatg

SEQ ID NO: 232 - DNA, 38 bp, Human papillomavirus
gatgcaaggtcgcatatgagtacactgctggacaacat

SEQ ID NO: 233 - DNA, 49 bp, Human papillomavirus
aattctaatacgactcactatagggagaaggtcatcttctgagctgtct

SEQ ID NO: 234 - DNA, 41 bp, Human papillomavirus
gatgcaaggtcgcatatgagtacactgctggacaacatgca

SEQ ID NO: 235 - DNA, 54 bp, Human papillomavirus
aattctaatacgactcactatagggagaagggtcacatccacagcaacaggtca

SEQ ID NO: 236 - DNA, 20 bp, Human papillomavirus
gtagggttacattgctatga

SEQ ID NO: 237 - DNA, 22 bp, Human papillomavirus
gtagggttacattgctatgagc

SEQ ID NO: 238 - DNA, 42 bp, Human papillomavirus
gatgcaaggtcgcatatgagtgacctgttgctgtggatgtga

SEQ ID NO: 239 - DNA, 50 bp, Human papillomavirus
aattctaatacgactcactatagggagaaggtacctgaatcgtccgccat

SEQ ID NO: 240 - DNA, 18 bp, Human papillomavirus
atwgtgtgtcccatctgc

SEQ ID NO: 241 - DNA, 39 bp, Human papillomavirus
gatgcaaggtcgcatatgagcatgccataaatgtataga

SEQ ID NO: 242 - DNA, 51 bp, Human papillomavirus
aattctaatacgactcactatagggagaaggcaccgcaggcaccttattaa

SEQ ID NO: 243 - DNA, 18 bp, Human papillomavirus
agaattagagaattaaga

SEQ ID NO: 244 - DNA, 41 bp, Human papillomavirus type 39
gatgcaaggtcgcatatgaggcagacgaccactacagcaaa

SEQ ID NO: 245 - DNA, 50 bp, Human papillomavirus type 39
aattctaatacgactcactatagggagaaggacaccgagtccgagtaata

SEQ ID NO: 246 - DNA, 19 bp, Human papillomavirus type 39
atagggacggggaaccact

SEQ ID NO: 247 - DNA, 39 bp, Human papillomavirus type 39
gatgcaaggtcgcatatgagtattactcggactcggtgt

SEQ ID NO: 248 - DNA, 52 bp, Human papillomavirus type 39
aattctaatacgactcactatagggagaaggcttgggtttctcttcgtgtta

SEQ ID NO: 249 - DNA, 20 bp, Human papillomavirus type 39
ggaccacaaaacgggaggac

SEQ ID NO: 250 - DNA, 40 bp, Human papillomavirus type 39
gatgcaaggtcgcatatgaggaaatagatgaacccgacca

SEQ ID NO: 251 - DNA, 51 bp, Human papillomavirus type 39
aattctaatacgactcactatagggagaagggcacaccacggacacacaaa

SEQ ID NO: 252 - DNA, 24 bp, Human papillomavirus type 39
tagccagacgggatgaaccacagc

SEQ ID NO: 253 - DNA, 41 bp, Human papillomavirus type 45
gatgcaaggtcgcatatgagaaccattgaacccagcagaaa

SEQ ID NO: 254 - DNA, 53 bp, Human papillomavirus type 45
aattctaatacgactcactatagggagaaggtctttcttgccgtgcctggtca

SEQ ID NO: 255 - DNA, 44 bp, Human papillomavirus type 45
gatgcaaggtcgcatatgaggaaaccattgaacccagcagaaaa

SEQ ID NO: 256 - DNA, 55 bp, Human papillomavirus type 45
aattctaatacgactcactatagggagaaggttgctatacttgtgtttccctacg

SEQ ID NO: 257 - DNA, 20 bp, Human papillomavirus type 45
gtaccgagggcagtgtaata

SEQ ID NO: 258 - DNA, 20 bp, Human papillomavirus type 45
ggacaaacgaagatttcaca

SEQ ID NO: 259 - DNA, 45 bp, Human papillomavirus type 45
gatgcaaggtcgcatatgaggttgacctgttgtgttaccagcaat

SEQ ID NO: 260 - DNA, 55 bp, Human papillomavirus type 45
aattctaatacgactcactatagggagaaggcaccacggacacacaaaggacaag

SEQ ID NO: 261 - DNA, 42 bp, Human papillomavirus type 45
gatgcaaggtcgcatatgagctgttgacctgttgtgttacga

SEQ ID NO: 262 - DNA, 53 bp, Human papillomavirus type 45
aattctaatacgactcactatagggagaaggccacggacacacaaaggacaag

SEQ ID NO: 263 - DNA, 40 bp, Human papillomavirus type 45
gatgcaaggtcgcatatgaggttgacctgttgtgttacga

SEQ ID NO: 264 - DNA, 51 bp, Human papillomavirus type 45
aattctaatacgactcactatagggagaaggacggacacacaaaggacaag

SEQ ID NO: 265 - DNA, 22 bp, Human papillomavirus type 45
gagtcagaggaggaaaacgatg

SEQ ID NO: 266 - DNA, 25 bp, Human papillomavirus type 45
aggaaaacgatgaagcagatggagt

SEQ ID NO: 267 - DNA, 25 bp, Human papillomavirus type 45
acaactaccagcccgacgagccgaa

SEQ ID NO: 268 - DNA, 40 bp, Human papillomavirus type 51
gatgcaaggtcgcatatgagggaggaggatgaagtagata

SEQ ID NO: 269 - DNA, 51 bp, Human papillomavirus type 51
aattctaatacgactcactatagggagaagggcccattaacatctgctgta

SEQ ID NO: 270 - DNA, 43 bp, Human papillomavirus type 51
gatgcaaggtcgcatatgagagaggaggaggatgaagtagata

SEQ ID NO: 271 - DNA, 51 bp, Human papillomavirus type 51
aattctaatacgactcactatagggagaaggacgggcaaaccaggcttagt

SEQ ID NO: 272 - DNA, 20 bp, Human papillomavirus type 51
gcaggtgttcaagtgtagta

SEQ ID NO: 273 - DNA, 24 bp, Human papillomavirus type 51
tggcagtggaaagcagtggagaca

SEQ ID NO: 274 - DNA, 44 bp, Human papillomavirus type 56
gatgcaaggtcgcatatgagttggggtgctggagacaaacatct

SEQ ID NO: 275 - DNA, 55 bp, Human papillomavirus type 56
aattctaatacgactcactatagggagaaggttcatcctcatcctcatcctctga

SEQ ID NO: 276 - DNA, 42 bp, Human papillomavirus type 56
gatgcaaggtcgcatatgagtggggtgctggagacaaacatc

SEQ ID NO: 277 - DNA, 53 bp, Human papillomavirus type 56
aattctaatacgactcactatagggagaaggcatcctcatcctcatcctctga

SEQ ID NO: 278 - DNA, 42 bp, Human papillomavirus type 56
gatgcaaggtcgcatatgagttggggtgctggagacaaacat

SEQ ID NO: 279 - DNA, 53 bp, Human papillomavirus type 56
aattctaatacgactcactatagggagaaggccacaaacttacactcacaaca

SEQ ID NO: 280 - DNA, 23 bp, Human papillomavirus type 56
aaagtaccaacgctgcaagacgt

SEQ ID NO: 281 - DNA, 24 bp, Human papillomavirus type 56
agaactaacacctcaaacagaaat

SEQ ID NO: 282 - DNA, 22 bp, Human papillomavirus type 56
agtaccaacgctgcaagacgtt

SEQ ID NO: 283 - DNA, 22 bp, Human papillomavirus type 56
ttggacagctcagaggatgagg

SEQ ID NO: 284 - DNA, 40 bp, Human papillomavirus type 56
gatgcaaggtcgcatatgaggattttccttatgcagtgtg

SEQ ID NO: 285 - DNA, 51 bp, Human papillomavirus type 56
aattctaatacgactcactatagggagaagggacatctgtagcaccttatt

SEQ ID NO: 286 - DNA, 20 bp, Human papillomavirus type 56
gactattcagtgtatggagc

SEQ ID NO: 287 - DNA, 22 bp, Human papillomavirus
caactgayctmyactgttatga

SEQ ID NO: 288 - DNA, 34 bp, Human papillomavirus
cgcatgcaactgayctmyactgttatgacatgcg

SEQ ID NO: 289 - DNA, 34 bp, Human papillomavirus
ccgtcgcaactgayctmyactgttatgacgacgg

SEQ ID NO: 290 - DNA, 34 bp, Human papillomavirus
ccaccccaactgayctmyactgttatgagggtgg

SEQ ID NO: 291 - DNA, 34 bp, Human papillomavirus
cgatcgcaactgayctmyactgttatgacgatcg

SEQ ID NO: 292 - DNA, 24 bp, Human papillomavirus
gaamcaactgacctaywctgctat

SEQ ID NO: 293 - DNA, 36 bp, Human papillomavirus
ccaagcgaamcaactgacctaywctgctatgcttgg

SEQ ID NO: 294 - DNA, 38 bp, Human papillomavirus
ccaagccgaamcaactgacctaywctgctatggcttgg

SEQ ID NO: 295 - DNA, 38 bp, Human papillomavirus
ccaagcggaamcaactgacctaywctgctatcgcttgg

SEQ ID NO: 296 - DNA, 36 bp, Human papillomavirus
ccagcggaamcaactgacctaywctgctatcgctgg

SEQ ID NO: 297 - DNA, 36 bp, Human papillomavirus
cgatcggaamcaactgacctaywctgctatcgatcg

SEQ ID NO: 298 - DNA, 18 bp, Human papillomavirus
aagacattattcagactc

SEQ ID NO: 299 - DNA, 30 bp, Human papillomavirus
ccaagcaagacattattcagactcgcttgg

SEQ ID NO: 300 - DNA, 30 bp, Human papillomavirus
cgcatgaagacattattcagactccatgcg

SEQ ID NO: 301 - DNA, 30 bp, Human papillomavirus
cccagcaagacattattcagactcgctggg

SEQ ID NO: 302 - DNA, 30 bp, Human papillomavirus
cgatcgaagacattattcagactccgatcg

SEQ ID NO: 303 - DNA, 24 bp, Human papillomavirus type 16
ccacaggagcgacccagaaagtta

SEQ ID NO: 304 - DNA, 22 bp, Human papillomavirus type 16
acggtttgttgtattgctgttc

SEQ ID NO: 305 - DNA, 20 bp, Human papillomavirus type 16
ccacaggagcgacccagaaa

SEQ ID NO: 306 - DNA, 20 bp, Human papillomavirus type 16
ggtttgttgtattgctgttc

SEQ ID NO: 307 - DNA, 23 bp, Human papillomavirus type 16
cagaggaggaggatgaaatagta

SEQ ID NO: 308 - DNA, 25 bp, Human papillomavirus type 16
gcacaaccgaagcgtagagtcacac

SEQ ID NO: 309 - DNA, 22 bp, Human papillomavirus type 16
cagaggaggaggatgaaataga

SEQ ID NO: 310 - DNA, 22 bp, Human papillomavirus type 16
gcacaaccgaagcgtagagtca

SEQ ID NO: 311 - DNA, 20 bp, Human papillomavirus type 18
acgatgaaatagatggagtt

SEQ ID NO: 312 - DNA, 20 bp, Human papillomavirus type 18
cacggacacacaaaggacag

SEQ ID NO: 313 - DNA, 22 bp, Human papillomavirus type 18
gaaaacgatgaaatagatggag

SEQ ID NO: 314 - DNA, 24 bp, Human papillomavirus type 18
acaccacggacacacaaaggacag

SEQ ID NO: 315 - DNA, 20 bp, Human papillomavirus type 18
ttccggttgaccttctatgt

SEQ ID NO: 316 - DNA, 20 bp, Human papillomavirus type 18
ggtcgtctgctgagctttct

SEQ ID NO: 317 - DNA, 21 bp, Human papillomavirus type 18
gcaagacatagaaataacctg

SEQ ID NO: 318 - DNA, 18 bp, Human papillomavirus type 18
acccagtgttagttagtt

SEQ ID NO: 319 - DNA, 20 bp, Human papillomavirus type 18
ggaaataccctacgatgaac

SEQ ID NO: 320 - DNA, 20 bp, Human papillomavirus type 18
ggacacaacggtctttgaca

SEQ ID NO: 321 - DNA, 22 bp, Human papillomavirus type 18
ggaaataccctacgatgaacta

SEQ ID NO: 322 - DNA, 22 bp, Human papillomavirus type 18
ctggacacaacggtctttgaca

SEQ ID NO: 323 - DNA, 20 bp, Human papillomavirus type 18
actgacctccactgttatga

SEQ ID NO: 324 - DNA, 20 bp, Human papillomavirus type 18
tatctacttgtgtgctctgt

SEQ ID NO: 325 - DNA, 24 bp, Human papillomavirus type 18
tgacctccactgttatgagcaatt

SEQ ID NO: 326 - DNA, 26 bp, Human papillomavirus type 18
tgcgaatatctacttgtgtgctctgt

SEQ ID NO: 327 - DNA, 18 bp, Human papillomavirus type 18
actgacctccactgttat

SEQ ID NO: 328 - DNA, 21 bp, Human papillomavirus type 18
cacgattccaaatgagcccat

SEQ ID NO: 329 - DNA, 22 bp, Human papillomavirus type 33
tatcctgaaccaactgacctat

SEQ ID NO: 330 - DNA, 19 bp, Human papillomavirus type 33
ttgacacataaacgaactg

SEQ ID NO: 331 - DNA, 20 bp, Human papillomavirus type 33
tcctgaaccaactgacctat

SEQ ID NO: 332 - DNA, 20 bp, Human papillomavirus type 33
cccataagtagttgctgtat

SEQ ID NO: 333 - DNA, 20 bp, Human papillomavirus type 33
gacctttgtgtcctcaagaa

SEQ ID NO: 334 - DNA, 20 bp, Human papillomavirus type 33
aggtcagttggttcaggata

SEQ ID NO: 335 - DNA, 20 bp, Human papillomavirus type 35
attacagcggagtgaggtat

SEQ ID NO: 336 - DNA, 20 bp, Human papillomavirus type 35
gtctttgcttttcaactgga

SEQ ID NO: 337 - DNA, 22 bp, Human papillomavirus type 35
tcagaggaggaggaagatacta

SEQ ID NO: 338 - DNA, 20 bp, Human papillomavirus type 35
gattatgctctctgtgaaca

SEQ ID NO: 339 - DNA, 20 bp, Human papillomavirus type 35
cccgaggcaactgacctata

SEQ ID NO: 340 - DNA, 20 bp, Human papillomavirus type 35
gtcaatgtgtgtgctctgta

SEQ ID NO: 341 - DNA, 22 bp, Human papillomavirus type 52
ttgtgtgaggtgctggaagaat

SEQ ID NO: 342 - DNA, 18 bp, Human papillomavirus type 52
ccctctcttctaatgttt

SEQ ID NO: 343 - DNA, 20 bp, Human papillomavirus type 52
gtgcctacgctttttatcta

SEQ ID NO: 344 - DNA, 22 bp, Human papillomavirus type 35
ggggtctccaacactctgaaca

SEQ ID NO: 345 - DNA, 18 bp, Human papillomavirus type 58
tcaggcgttggagacatc

SEQ ID NO: 346 - DNA, 18 bp, Human papillomavirus type 58
agcaatcgtaagcacact

SEQ ID NO: 347 - DNA, 18 bp, Human papillomavirus type 58
tctgtgcatgaaatcgaa

SEQ ID NO: 348 - DNA, 18 bp, Human papillomavirus type 58
agcacactttacatactg

SEQ ID NO: 349 - DNA, 18 bp, Human papillomavirus
tacactgctggacaacat

SEQ ID NO: 350 - DNA, 18 bp, Human papillomavirus
tcatcttctgagctgtct

SEQ ID NO: 351 - DNA, 21 bp, Human papillomavirus
tacactgctggacaacatgca

SEQ ID NO: 352 - DNA, 23 bp, Human papillomavirus
gtcacatccacagcaacaggtca

SEQ ID NO: 353 - DNA, 22 bp, Human papillomavirus
tgacctgttgctgtggatgtga

SEQ ID NO: 354 - DNA, 19 bp, Human papillomavirus
tacctgaatcgtccgccat

SEQ ID NO: 355 - DNA, 19 bp, Human papillomavirus
catgccataaatgtataga

SEQ ID NO: 356 - DNA, 20 bp, Human papillomavirus
caccgcaggcaccttattaa

SEQ ID NO: 357 - DNA, 21 bp, Human papillomavirus
gcagacgaccactacagcaaa

SEQ ID NO: 358 - DNA, 19 bp, Human papillomavirus
acaccgagtccgagtaata

SEQ ID NO: 359 - DNA, 19 bp, Human papillomavirus
tattactcggactcggtgt

SEQ ID NO: 360 - DNA, 21 bp, Human papillomavirus
cttgggtttctcttcgtgtta

SEQ ID NO: 361 - DNA, 20 bp, Human papillomavirus
gaaatagatgaacccgacca

SEQ ID NO: 362 - DNA, 20 bp, Human papillomavirus
gcacaccacggacacacaaa

SEQ ID NO: 363 - DNA, 21 bp, Human papillomavirus type 45
aaccattgaacccagcagaaa

SEQ ID NO: 364 - DNA, 22 bp, Human papillomavirus type 45
tctttcttgccgtgcctggtca

SEQ ID NO: 365 - DNA, 24 bp, Human papillomavirus type 45
gaaaccattgaacccagcagaaaa

SEQ ID NO: 366 - DNA, 24 bp, Human papillomavirus type 45
ttgctatacttgtgtttccctacg

SEQ ID NO: 367 - DNA, 25 bp, Human papillomavirus type 45
gttgacctgttgtgttaccagcaat

SEQ ID NO: 368 - DNA, 24 bp, Human papillomavirus type 45
caccacggacacacaaaggacaag

SEQ ID NO: 369 - DNA, 22 bp, Human papillomavirus type 45
ctgttgacctgttgtgttacga

SEQ ID NO: 370 - DNA, 22 bp, Human papillomavirus type 45
ccacggacacacaaaggacaag

SEQ ID NO: 371 - DNA, 20 bp, Human papillomavirus type 45
gttgacctgttgtgttacga

SEQ ID NO: 372 - DNA, 20 bp, Human papillomavirus type 45
acggacacacaaaggacaag

SEQ ID NO: 373 - DNA, 20 bp, Human papillomavirus type 51
ggaggaggatgaagtagata

SEQ ID NO: 374 - DNA, 20 bp, Human papillomavirus type 51
gcccattaacatctgctgta

SEQ ID NO: 375 - DNA, 23 bp, Human papillomavirus type 51
agaggaggaggatgaagtagata

SEQ ID NO: 376 - DNA, 20 bp, Human papillomavirus type 51
acgggcaaaccaggcttagt

SEQ ID NO: 377 - DNA, 24 bp, Human papillomavirus type 56
ttggggtgctggagacaaacatct

SEQ ID NO: 378 - DNA, 24 bp, Human papillomavirus type 56
ttcatcctcatcctcatcctctga

SEQ ID NO: 379 - DNA, 22 bp, Human papillomavirus type 56
tggggtgctggagacaaacatc

SEQ ID NO: 380 - DNA, 22 bp, Human papillomavirus type 56
catcctcatcctcatcctctga

SEQ ID NO: 381 - DNA, 22 bp, Human papillomavirus type 56
ttggggtgctggagacaaacat

SEQ ID NO: 382 - DNA, 22 bp, Human papillomavirus type 56
ccacaaacttacactcacaaca

SEQ ID NO: 383 - DNA, 20 bp, Human papillomavirus type 56
gattttccttatgcagtgtg

SEQ ID NO: 384 - DNA, 20 bp, Human papillomavirus type 56
gacatctgtagcaccttatt

SEQ ID NO: 385 - DNA, 31 bp, Artificial
  T7 promoter sequence
aattctaatacgactcactatagggagaagg

SEQ ID NO: 386 - DNA, 25 bp, Artificial
  T7 promoter sequence
aattctaatacgactcactataggg

SEQ ID NO: 387 - DNA, 20 bp, Artificial
  capable of hybridising to generic ECL (RTM) probe
gatgcaaggtcgcatatgag